(19) United States
(12) Patent Application Publication
(10) Pub. No.: US 2026/0218142 A1
(43) Pub. Date: Jul. 30, 2026

(54) ENGINEERED NUCLEASE WITH HIGH SALT TOLERANCE

(71) Applicant: WUXI BIOLOGICS IRELAND LIMITED, Dundalk (IE)

(72) Inventors: Shanhui LIAO, Shanghai (CN); Cunjia QIU, Shanghai (CN); Xiaoyue CHEN, Shanghai (CN); Jiexing CAI, Shanghai (CN); Weichang ZHOU, Shanghai (CN)

(73) Assignee: WUXI BIOLOGICS IRELAND LIMITED, Dundalk (IE)

(21) Appl. No.: 19/142,026

(22) PCT Filed: Dec. 20, 2023
(86) PCT No.: PCT/CN2023/140228

§ 371 Date: Jun. 20, 2025

(30) Foreign Application Priority Data
Dec. 20, 2022 (WO) PCT/CN2022/140354

(51) Int. Cl. C12N 9/22; C12N 7/02; C12N 15/52; C12N 15/86

(52) U.S. Cl. CPC C12N 9/22; C12N 7/025; C12N 15/52; C12N 15/86; C12N 2830/34; C12Y 301/30002

(57) ABSTRACT

Provided is an engineered nuclease with high salt tolerance. The polypeptide comprises one or more mutations so that the polypeptide possesses more positive charged surface area in its three-dimensional structure. The polypeptide has at least 60% nuclease activity under a solution ionic strength more than 200 mM as compared to the nuclease having the sequence of SEQ ID NO: 1.

### TECHNICAL FIELD

The present disclosure concerns the field of protein engineering and recombinant expression technologies. It inter alia pertains to altered biochemical properties of a nuclease from Serratia marcescens by combination of amino acid residues mutations in protein primary sequence, to enhance enzyme activity of mutants under high salt concentration.

### BACKGROUND

Nucleases are kinds of enzymes responsible for cleaving phosphodiester bonds of nucleic acids in an endo-/exo-way. Serratia marcescens nuclease A (Uniprot: P13717) has been identified as a non-specific nuclease, which catalyzes the hydrolysis of both single stranded/double stranded DNA and RNA by cleaving the phosphodiester bond. Crystal structure indicated that this enzyme is a dimeric Mg²⁺ dependent nuclease, and a water cluster is essential for its activity. It has been widely used to remove nucleic acid in protein production and pharmaceutical virus production. This enzyme was also commercialized under the name of Benzonase® by EMD Millipore Corp.

Although Serratia marcescens nuclease A has been widely used in industry and R & D fields, inhibition by high salt concentration is a significant defect of this enzyme. The high salt unfavorable property of this enzyme limits its usage and increases additional buffer change steps in some pharmaceutical production. The enzyme activity is significantly inhibited under solution ionic strengths of >200 mM. however, recent researches indicated high salt buffer is better for virus production. Thus, a high salt tolerant Serratia marcescens nuclease A should be more useful in many applications under high salt condition.

### SUMMARY

Disclosed herein includes polypeptides having nuclease activity (hereinafter “nucleases” or “nuclease polypeptides”), polynucleotides comprising the coding sequences for these polypeptides, and methods for making and using these polypeptides and polynucleotides. Also provided herein are compositions and kits comprising one or more of the nuclease polypeptides disclosed herein, one or more of the nuclease-coding polynucleotides, and any combination thereof.

Disclosed herein includes synthetic or recombinant polypeptide derived from Serratia marcescens nuclease A, and having high salt tolerance. In some embodiments, the polypeptide comprises one or more mutations so that the polypeptide possesses more positive charged surface area in its three-dimensional structure.

In some embodiments, the polypeptide has at least 60%, at least 70%, at least 80%, at least 90%, or at least 95% nuclease activity under a solution ionic strength more than 200 mM as compared to the nuclease having the sequence of SEQ ID NO: 1 or the mature polypeptide thereof. In some embodiments, the ion is a monovalent ion or divalent ion. In some embodiments, the solution ionic strength is more than 300 mM. In some embodiments, the solution ionic strength is more than 400 mM. In some embodiments, the solution ionic strength is more than 500 mM.

In some embodiments, the polypeptide comprises an amino acid sequence having at least 70%, at least 80%, at least 85%, at least 90%, at least 95%, or at least 99% sequence identity to SEQ ID NO: 1 or the mature polypeptide thereof. In some embodiments, the polypeptide has nuclease activity, and wherein the polypeptide comprises mutations at 1, 2, 3, 4, 5, 6, 7 or more or all sites of S74, N79, A95, T98, N101, A102, S137, D138, Q141, D149, D156. The mutations include modification, substitution or deletion of amino acids.

In some embodiments, the polypeptide:
(a) comprises an amino acid sequence having at least 70% identity to SEQ ID NO: 1 or the mature polypeptide thereof and has mutations at 1, 2, 3, 4, 5, 6, 7 or more or all sites of S74, N79, A95, T98, N101, A102, S137, D138, Q141, D149, D156, or
(b) comprises an amino acid sequence having at least 70%, at least 80%, at least 85%, at least 90%, at least 95%, or at least 99% sequence identity to the sequence of (a) and has the mutation at the site as described in (a) and retains nuclease activity.

In some embodiments, the mutation of N79 is a mutation to a non polar amino acids such as alanine, valine, isoleucine, proline, phenylalanine, tryptophan methionine, or glycine, or to an uncharged polar amino acid such as serine, threonine, glutamine, tyrosine, or cysteine, or to a polar amino acid with positive charges such as histidine, lysine or arginine.

In some embodiments, the mutation of A95 is a mutation to a non polar amino acids such as valine, isoleucine, proline, phenylalanine, tryptophan methionine, or glycine, or to an uncharged polar amino acid such as serine, threonine, glutamine, asparagine, tyrosine, or cysteine, or to a polar amino acid with positive charges such as histidine, lysine or arginine.

In some embodiments, the mutation of A102 is a mutation to a non polar amino acids such as valine, isoleucine, proline, phenylalanine, tryptophan methionine, or glycine, or to an uncharged polar amino acid such as serine, threonine, glutamine, asparagine, tyrosine, or cysteine, or to a polar amino acid with positive charges such as histidine, lysine or arginine.

In some embodiments, the mutation of D149 is a mutation to a non polar amino acids such as alanine, valine, isoleucine, proline, phenylalanine, tryptophan methionine, or glycine, or to an uncharged polar amino acid such as serine, threonine, glutamine, asparagine, tyrosine, or cysteine, or to a polar amino acid with positive charges such as histidine, lysine or arginine.

In some embodiments, the mutation of S74, N79, A95, T98, N101, A102, S137, D138, Q141, D149, D156 is a mutation to a polar amino acid with positive charges, preferably histidine, lysine or arginine, more preferably lysine or arginine.

In some embodiments, the polypeptide comprises an amino acid sequence having at least 70% identity to SEQ ID NO: 1 or the mature polypeptide thereof and has mutations at at least 4 sites selected from the group consisting of S74, N79, A95, T98, N101, A102, S137, D138, Q141, D149, D156.

In some embodiments, the polypeptide comprises an amino acid sequence having at least 70% identity to SEQ ID NO: 1 or the mature polypeptide thereof and has a mutation at A95 or D149 and at least 3 sites selected from the group consisting of S74, N79, A95, T98, N101, A102, S137, D138, Q141, D156. Preferably, the polypeptide has mutations at A95 and D149 and at least 2 sites selected from the group consisting of S74, N79, T98, N101, A102, S137, D138, Q141, D156.

In some embodiments, the polypeptide comprises an amino acid sequence having at least 70% identity to SEQ ID NO: 1 or the mature polypeptide thereof, and has mutations at at least three sites selected from the group consisting of N79, A95, A102, and D149 and optionally at least one site selected from the group consisting of S74, T98, N101, S137, D138, Q141, D156.

In some embodiments, the polypeptide comprises an amino acid sequence having at least 70% identity to SEQ ID NO: 1 or the mature polypeptide thereof, and (1) has a mutation at A95, A102, and D149 and optionally at least one sites selected from the group consisting of S74, N79, T98, N101, S137, D138, Q141, D156; (2) has a mutation at N79, A102, and D149 and optionally at least one sites selected from the group consisting of S74, A95, T98, N101, S137, D138, Q141, D156; (3) has a mutation at N79, A95, and D149 and optionally at least one sites selected from the group consisting of S74, T98, N101, A102, S137, D138, Q141, D156; or (4) has a mutation at N79, A95, and A102 and optionally at least one sites selected from the group consisting of S74, T98, N101, A102, S137, D138, Q141, D149, D156.

In some embodiments, the polypeptide comprises an amino acid sequence having at least 70% identity to SEQ ID NO: 1 or the mature polypeptide thereof, and has mutations at N79, A95, A102, and D149 and optionally at least one site selected from the group consisting of S74, T98, N101, S137, D138, Q141, D156. In some embodiments, the polypeptide has mutations at N79, A95, A102, and D149 and optionally at one or two sites selected from the group consisting of Q141 and N101.

In some embodiments, the polypeptide comprises an amino acid sequence having at least 70% identity to SEQ ID NO: 1 or the mature polypeptide thereof, and has mutations at A95, N101, Q141, D149 and optionally at least 3 sites selected from the group consisting of S74, N79, T98, A102, S137, D138, D156. In some embodiments, the polypeptide has mutations at A95, N101, Q141, D149 and optionally at S74, N79, and T98.

In some embodiments, the polypeptide can be a polypeptide comprising an amino acid sequence having at least 70%, at least 80%, at least 85%, at least 90%, at least 95%, or at least 99% sequence identity to SEQ ID NO: 1 or the mature polypeptide thereof, where the polypeptide has nuclease activity under high solution ionic strength, and where the polypeptide comprises one or more mutations of (1) N79K or N79R, (2) A95K or A95R, (3) A102K or A102R, (4) D149K or D149R, (5) S74K, (6) T98K, (7) N101K, (8) S137K, (9) D138K, (10) Q141K, and (11) D156R; preferably one or more mutations of S74K, N79K, A95K, T98K, N101K, A102K, S137K, D138K, Q141K, D149K, and D156R.

In some embodiments, the polypeptide:

(i) comprises an amino acid sequence having at least 70% identity to SEQ ID NO: 1 or the mature polypeptide thereof and has a mutation selected from the group consisting of (1) N79K or N79R, (2) A95K or A95R, (3) A102K or A102R, (4) D149K or D149R, (5) S74K, (6) T98K, (7) N101K, (8) S137K, (9) D138K, (10) Q141K, and (11) D156R, preferably a mutation selected from the group consisting of S74K, N79K, A95K, T98K, N101K, A102K, S137K, D138K, Q141K, D149K, D156R, or
(ii) comprises an amino acid sequence having at least 70%, 80%, 85%, 90%, 95%, or 99% sequence identity to the sequence of (i) and has the mutation as described in (i) and retains nuclease activity.

In some embodiments, the polypeptide comprises an amino acid sequence having at least 70% identity to SEQ ID NO: 1 or the mature polypeptide thereof and has at least 4 mutations selected from the group consisting of (1) N79K or N79R, (2) A95K or A95R, (3) A102K or A102R, (4) D149K or D149R, (5) S74K, (6) T98K, (7) N101K, (8) S137K, (9) D138K, (10) Q141K, and (11) D156R, preferably the group consisting of S74K, N79K, A95K, T98K, N101K, A102K, S137K, D138K, Q141K, D149K, and D156R.

In some embodiments, the polypeptide comprises an amino acid sequence having at least 70% identity to SEQ ID NO: 1 or the mature polypeptide thereof and has (a) the mutation of A95K or A95R and at least 3 (or at least 4, at least 5, at least 6, at least 7 or more) mutations selected from the group consisting of (1) N79K or N79R, (2) A102K or A102R, (3) D149K or D149R, (4) S74K, (5) T98K, (6) N101K, (7) S137K, (8) D138K, (9) Q141K, and (10) D156R, preferably the group consisting of S74K, N79K, T98K, N101K, A102K, S137K, D138K, Q141K, D149K, and D156R; or (b) the mutation of D149K or D149R and at least 3 (or at least 4, at least 5, at least 6, at least 7 or more) mutations selected from the group consisting of (1) N79K or N79R, (2) A95K or A95R, (3) A102K or A102R, (4) S74K, (5) T98K, (6) N101K, (7) S137K, (8) D138K, (9) Q141K, and (10) D156R preferably the group consisting of S74K, N79K, A95K, T98K, N101K, A102K, S137K, D138K, Q141K, and D156R. More preferably, the polypeptide has mutations of (i) A95K or A95R and (ii) D149K or D149R and at least 2 (or at least 3, at least 4, at least 5, at least 6 or more) mutations selected from the group consisting of (1) N79K or N79R, (2) A102K or A102R, (3) S74K, (4) T98K, (5) N101K, (6) S137K, (7) D138K, (8) Q141K, and (9) D156R, preferably the group consisting of S74K, N79K, T98K, N101K, A102K, S137K, D138K, Q141K, and D156R.

In some embodiments, the polypeptide comprises an amino acid sequence having at least 70% identity to SEQ ID NO: 1 or the mature polypeptide thereof and has at least three mutations selected from the group consisting of (1) N79K or N79R, (2) A95K or A95R, (3) A102K or A102R, (4) D149K or D149R, and optionally has a mutation selected from the group consisting of S74K, T98K, N101K, S137K, D138K, Q141K, and D156R.

In some embodiments, the polypeptide comprises an amino acid sequence having at least 70% identity to SEQ ID NO: 1 or the mature polypeptide thereof, and has mutations of (i) N79K, A95K, A102K, D149K, or (ii) N79R, A95R, A102R and D149R, and optionally has a mutation selected from the group consisting of S74K, T98K, N101K, S137K, D138K, Q141K, and D156R. In some embodiments, the polypeptide has mutations of (i) N79K, A95K, A102K, and D149K or (ii) N79R, A95R, A102R and D149R, and optionally has a mutation selected from the group consisting of Q141K and N101K.

In some embodiments, the polypeptide comprises an amino acid sequence having at least 70% identity to SEQ ID NO: 1 or the mature polypeptide thereof, and has mutations of A95K, N101K, Q141K, D149K and optionally has a mutation selected from the group consisting of S74K, N79K, T98K, A102K, S137K, D138K, and D156R. In some embodiments, the polypeptide has mutations of A95K, N101K, Q141K, D149K and optionally mutations of S74K, N79K, and T98K.

In some embodiments, the polypeptide comprises an amino acid sequence having at least 70% identity to SEQ ID NO: 1 or the mature polypeptide thereof, and has mutations of S74K, N79K, A95K, T98K, N101K, A102K, S137K, D138K, Q141K, D149K, and D156R.

In some embodiments, the optimal temperature of the polypeptide is between 30° C. to 60° C. In some embodiments, the optimal pH of the polypeptide is between pH 4 to pH 11. In some embodiments, the polypeptide comprises no signal sequence. In some embodiments, the polypeptide further comprises a signal sequence. In some embodiments, the signal sequence is a heterologous sequence or a native signal sequence.

Also disclosed herein includes a composition or kit comprising one or more of the polypeptides disclosed herein. In some embodiments, the composition is a reaction mixture, a detergent composition, a detergent additive, a food, a food supplement, a feed supplement, a feed, a pharmaceutical composition, a fermentation product, a fermentation intermediate, a fermentation downstream reaction mixture, or a combination thereof. In some embodiments, the reaction mixture comprising one or more of the polypeptides is for expression or purification of protein or virus vector. For example, the polypeptide can be a polypeptide comprising an amino acid sequence having at least 70% sequence identity to SEQ ID NO: 1 or the mature polypeptide thereof, where the polypeptide has nuclease activity under high solution ionic strength, and where the polypeptide comprises one or more mutations of S74K, N79K, A95K, T98K, N101K, A102K, S137K, D138K, Q141K, D149K, and D156R.

Also disclosed herein includes a nucleic acid comprising: (1) the sequence that encodes any one of the polypeptides disclosed herein, or a complementary sequence thereof, or (2) a sequence with at least 50%, 60%, 70%, 80%, or 90% identity with (1).

Also disclosed herein includes nucleic acid constructs comprising the nucleic acid sequences described herein. In one or more embodiments, the nucleic acid construct is a cloning vector, expression vector, or recombinant vector. In one or more embodiments, the nucleic acid sequence is operably linked to an expression control sequence. In some embodiments, the expression vector comprises a viral vector, a plasmid, a phage, a phagemid, a cosmid, a fosmid, a bacteriophage, an artificial chromosome, or a combination thereof.

Disclosed herein includes a recombinant cell comprising one or more of the polypeptides disclosed herein, one or more nucleic acid that encodes any one of the polypeptides disclosed herein, one or more nucleic acid constructs comprising the polynucleotide sequence of the nucleic acid, or a combination thereof. In some embodiments, the nucleic acid is a part of a chromosome of the recombinant cell. In some embodiments, the recombinant cell is a bacterial cell, a mammalian cell, a fungal cell, a yeast cell, an insect cell, or a plant cell. For example, the polypeptide can be a polypeptide comprising an amino acid sequence having at least 70% sequence identity to SEQ ID NO: 1 or the mature polypeptide thereof, where the polypeptide has nuclease activity under high solution ionic strength, and where the polypeptide comprises one or more mutations of S74K, N79K, A95K, T98K, N101K, A102K, S137K, D138K, Q141K, D149K, and D156R.

Disclosed herein includes a method of producing a polypeptide having nuclease activity under high solution ionic strength. The method, in some embodiments, comprises: expressing the nucleic acid that encodes any one of the polypeptides disclosed herein under conditions that allow expression of the polypeptide, thereby producing recombinant polypeptide having nuclease activity, wherein the nucleic acid is operably linked to a promoter. In some embodiments, the nucleic acid is present in an expression vector. In some embodiments, the nucleic acid is present in a host cell to allow expression of the polypeptide. In some embodiments, the nucleic acid is present in a chromosome of the host cell. In some embodiments, the host cell is a cell from an organism selected from the group consisting of Pichia pastoris, Bacillus subtilis, Pseudomonas fluorescens, Myceliopthora thermophile fungus, Tricodermea reesei, Escherichia coli, Bacillus licheniformis, Aspergillus niger, Schizosaccharomyces pombe, and. Sacaramyces cerevisiae. In some embodiments, the nucleic acid is present by an in vitro expression system. The polypeptide can be a polypeptide comprising an amino acid sequence having at least 70% sequence identity to SEQ ID NO: 1 or the mature polypeptide thereof, where the polypeptide has nuclease activity under high solution ionic strength, and where the polypeptide comprises one or more mutations of S74K, N79K, A95K, T98K, N101K, A102K, S137K, D138K, Q141K, D149K, and D156R.

Disclosed herein includes a method for degrading a polynucleotide, comprising contacting a polynucleotide molecule with one or more of the polypeptides disclosed herein, thereby degrading the polynucleotide molecule. For example, the polypeptide can be a polypeptide comprising an amino acid sequence having at least 70% sequence identity to SEQ ID NO: 1 or the mature polypeptide thereof, where the polypeptide has nuclease activity under high solution ionic strength, and where the polypeptide comprises one or more mutations of S74K, N79K, A95K, T98K, N101K, A102K, S137K, D138K, Q141K, D149K, and D156R. In some embodiments, the polynucleotide molecule is a DNA molecule or a RNA molecule. In some embodiments, the contacting occurs at pH 4 to pH 11. In some embodiments, the reaction mixture has a temperature at about 10° C. to about 70° C. In some embodiments, the contacting occurs at 30° C. to 60° C.

Also disclosed herein include a method for degrading DNA or RNA during cell lysis. The method comprises, in some embodiments, lysing a host cell of interest; and adding a polypeptide disclosed herein under conditions that allow degradation of DNA or RNA by the polypeptide. The host cell can be, for example, a bacterial cell, a mammalian cell, a fungal cell, a yeast cell, a plant cell, or an insect cell.

Also disclosed herein include a method for degrading DNA or RNA during virus vector production. The method, in some embodiments, comprises culturing a host cell, wherein the host cell comprises a virus vector of interest; and expressing or adding one or more of the polypeptides disclosed herein under conditions that allow degradation of DNA or RNA other than the virus vector by the one or more polypeptides. In some embodiments, the host cell is a bacterial cell, a mammalian cell, a fungal cell, a yeast cell, a plant cell, or an insect cell. In some embodiments, the polypeptide is expressed from an expression vector present in the host cell or the polypeptide is encoded by a nucleic acid sequence in a chromosome of the host cell. In some embodiments, the virus is adeno-associated virus (AAV), or lentivirus.

In some embodiments, the polypeptide is expressed by cells that do not express the virus vector of interest. In some embodiments, expression of the virus vector of interest and the polypeptide is inducible or non-inducible. In some embodiments, one or more of the polypeptides disclosed herein are added externally. For example, the polypeptide can be a polypeptide comprising an amino acid sequence having at least 70% sequence identity to SEQ ID NO: 1 or the mature polypeptide thereof, where the polypeptide has nuclease activity under high solution ionic strength, and where the polypeptide comprises one or more mutations of S74K, N79K, A95K, T98K, N101K, A102K, S137K, D138K, Q141K, D149K, and D156R.

Also disclosed herein includes a method for degrading DNA or RNA during protein production. The method, in some embodiments, comprises culturing a host cell, wherein the host cell comprises a nucleic acid encoding a protein of interest; and expressing or adding one or more of the polypeptides disclosed herein under conditions that allow degradation of DNA or RNA by the one or more polypeptides. In some embodiments, the host cell is a bacterial cell, a mammalian cell, a fungal cell, a yeast cell, a plant cell, or an insect cell. In some embodiments, the polypeptide is expressed from an expression vector present in the host cell or the polypeptide is encoded by a nucleic acid sequence in a chromosome of the host cell.

In some embodiments, the polypeptide is expressed by cells that do not express the protein of interest. In some embodiments, expression of one or more of the protein of interest and the polypeptide is inducible or non-inducible. In some embodiments, one or more of the polypeptides disclosed herein are added externally. For example, the polypeptide can be a polypeptide comprising an amino acid sequence having at least 70% sequence identity to SEQ ID NO: 1 or the mature polypeptide thereof, where the polypeptide has nuclease activity under high solution ionic strength, and where the polypeptide comprises one or more mutations of S74K, N79K, A95K, T98K, N101K, A102K, S137K, D138K, Q141K, D149K, and D156R.

Also disclose herein include a reaction mixture, wherein the reaction mixture comprises: (a) one or more of the polypeptides disclosed herein, (b) one or more nucleic acid molecules, and (c) an aqueous solution wherein the polypeptide hydrolyzes the one or more nucleic acid molecules. In some embodiments, the one or more nucleic acid molecules comprise single-stranded DNA molecules, double-stranded DNA molecules, single-stranded RNA molecules, double-stranded RNA molecules, or any combination thereof. In some embodiments, the one or more nucleic acid molecules are from a host cell for protein production. In some embodiments, the polypeptide is expressed in a host cell selected from the group consisting of a bacterial cell, a mammalian cell, a fungal cell, a yeast cell, and an insect cell. In some embodiments, the reaction mixture has a temperature at about 10° C. to about 70° C. In some embodiments, the reaction mixture has a temperature at about 30° C. to about 60° C. In some embodiments, the reaction mixture is at about pH 4 to about pH 11. In some embodiments, the reaction mixture is at about pH 7 to about pH 9. In some embodiments, the aqueous solution is a detergent composition, a detergent additive, a food, a food supplement, a feed supplement, a feed, a pharmaceutical composition, a fermentation product, a fermentation intermediate, a fermentation downstream reaction mixture, a product from protein production process, an intermediate from protein production process, a protein purification solution, or a combination thereof. For example, the polypeptide can be a polypeptide comprising an amino acid sequence having at least 70% sequence identity to SEQ ID NO: 1 or the mature polypeptide thereof, where the polypeptide has nuclease activity under high solution ionic strength, and where the polypeptide comprises one or more mutations of S74K, N79K, A95K, T98K, N101K, A102K, S137K, D138K, Q141K, D149K, and D156R.

Disclosed herein include a method for degrading DNA or RNA in a protein production mixture. The method comprises, in some embodiments, culturing a host cell which comprises a nucleic acid encoding a protein of interest; and expressing one or more of the polypeptides disclosed herein under conditions that allow degradation of DNA or RNA by the polypeptide. The expression of the polypeptide can be delayed, in some embodiments, until after the production of the protein of interest. In some embodiments, the expression of the polypeptide is not delayed. The expression of the polypeptide can start before, after, or at the same time with the start of the expression of the protein of interest. The host cell can be, for example, a bacterial cell, a mammalian cell, a fungal cell, a yeast cell, a plant cell, or an insect cell. In some embodiments, the polypeptide is expressed from an expression vector present in the host cell or the polypeptide is encoded by a nucleic acid sequence in a chromosome of the host cell. In some embodiments, the polypeptide is expressed by cells that do not express the protein of interest. The expression of one or more of the protein of interest and the polypeptides can be inducible or non-inducible.

### BRIEF DESCRIPTION OF THE DRAWINGS

FIG. 1. Electrostatic potential surface of wild type NucA from Serratia marcescens (left) and engineered NucA (HighSalt NucA) with more positive charged residues (Right). The white color represents non-charged surface, and the dark shadowed region represents the charged surfaces. Comparing to the wild type NucA, the engineered HighSalt NucA possesses more positive charged surface area.

FIG. 2. Digestion of lambda DNA by wild type NucA from Serratia marcescens and engineered NucA (HighSalt NucA) under different NaCl concentrations. Enzyme activity of wild type NucA is greatly inhibited when salt concentration increases upto 300 mM. Engineered HighSalt NucA can efficiently digest lambda DNA under 500 mM NaCl.

FIG. 3. Digestion of plasmid DNA by wild type NucA from Serratia marcescens and engineered NucA (HighSalt NucA) under different NaCl concentrations. Enzyme activity of wild type NucA is greatly inhibited when salt concentration upto 300 mM. Engineered HighSalt NucA can efficiently digest plasmid DNA under 500 mM NaCl.

FIG. 4. Digestion of total RNA extracted from CHO cells by wild type NucA from Serratia marcescens and engineered NucA (HighSalt NucA) under different NaCl concentrations. Enzyme activity of wild type NucA is greatly inhibited when salt concentration upto 300 mM. Engineered HighSalt NucA can efficiently digest RNA under 500 mM NaCl.

FIG. 5. Digestion of single stranded DNA (ssDNA) by wild type NucA from Serratia marcescens and engineered NucA (HighSalt NucA) under different NaCl concentrations. Enzyme activity of wild type NucA is greatly inhibited when salt concentration upto 300 mM. Engineered HighSalt NucA can efficiently digest ssDNA under 500 mM NaCl.

FIG. 6. Inhibition effects of monovalent salts (left: NaCl; right: KCl) on HighSalt NucA and wild type nuclease A.

FIG. 7. Inhibition effects of divalent salts (left: MgCl₂; right: MnCl₂) on HighSalt NucA and wild type nuclease A.

FIG. 8. Inhibition effects of (NH₄) ₂SO₄ on HighSalt NucA and wild type nuclease A.

FIG. 9. Inhibition effects of Na₂HPO₄ on HighSalt NucA and wild type nuclease A.

FIG. 10. The salt tolerance of wild type NucA, HighSalt NucA and nineteen mutants with single mutation on N79 residue.

FIG. 11. The salt tolerance of wild type NucA, HighSalt NucA and nineteen mutants with single mutation on A95 residue.

FIG. 12. The salt tolerance of wild type NucA, HighSalt NucA and nineteen mutants with single mutation on A102 residue.

FIG. 13. The salt tolerance of wild type NucA, HighSalt NucA and nineteen mutants with single mutation on D149 residue.

FIG. 14. The salt tolerance of wild type NucA, HighSalt NucA and mutants with mutations of combination positive residues

### DETAILED DESCRIPTION

All patents, applications, published applications and other publications referred to herein are incorporated by reference for the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

Unless defined otherwise, all technical and scientific terms used herein have the same meaning as is commonly understood by one of ordinary skill in the art to which this disclosure belongs. In the event that there is a plurality of definitions for a term herein, those in this section prevail unless stated otherwise.

As used herein, the singular forms “a”, “an”, and “the” include plural references unless indicated otherwise, expressly or by context. For example, “a” dimer includes one or more dimers, unless indicated otherwise, expressly or by context.

The term “amplification” (“a polymerase extension reaction”) means that the number of copies of a polynucleotide is increased.

As used herein, “sequence identity” or “identity” in the context of two protein sequences (or nucleotide sequences) includes reference to the residues in the two sequences which are the same when aligned for maximum correspondence over a specified comparison window.

Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), such program is within the skill in the art, such as “NEEDLE”. The preferred alignment for the purpose of this description is that alignment, from which the highest sequence identity can be determined.

After aligning two sequences, in a second step, an identity value is determined from the alignment produced. For purposes of this description, percent identity is calculated by: % identity=(identical residues/length of the alignment region which is showing the respective sequence of this description over its complete length)*100.

Thus, sequence identity in relation to comparison of two amino acid sequences according to this embodiment is calculated by dividing the number of identical residues by the length of the alignment region which is showing the respective sequence of this description over its complete length. This value is multiplied with 100 to give “% identity”.

For calculating the percent identity of two DNA sequences the same applies as for the calculation of percent identity of two amino acid sequences with some specifications.

For DNA sequences encoding for a protein the pairwise alignment shall be made over the complete length of the coding region from start to stop codon excluding introns. Introns, present in the other sequence, so the sequence to which the sequence of this description is compared, may also be removed for the pairwise alignment. Percent identity is then calculated by: % identity=(identical residues/length of the alignment region which is showing the coding region of the sequence of this description from start to stop codon excluding introns over its complete length)*100.

Sequences, having identical or similar regions with a sequence of this description, and which shall be compared with a sequence of this description to determine % identity, can easily be identified by various ways that are within the skill in the art, for instance, using publicly available computer methods and programs such as BLAST, available for example at NCBI.

Variants of the parent enzyme molecules may have an amino acid sequence which is at least n percent identical to the amino acid sequence of the respective parent enzyme having enzymatic activity with n being an integer between 50 and 100, preferably 50, 55, 60, 65, 70, 75, 80, 85, 90, 91, 92, 93, 94, 95, 96, 97, 98 or 99 compared to the full length polypeptide sequence. Preferably, variant enzymes which are n percent identical when compared to a parent enzyme, have enzymatic activity.

Enzyme variants may be defined by their sequence similarity when compared to a parent enzyme. Sequence similarity usually is provided as “% sequence similarity” or “% similarity”. For calculating sequence similarity in a first step a sequence alignment has to be generated as described above. In a second step, the percent-similarity has to be calculated, whereas percent sequence similarity takes into account that defined sets of amino acids share similar properties, e.g., by their size, by their hydrophobicity, by their charge, or by other characteristics. Herein, the exchange of one amino acid with a similar amino acid is called “conservative mutation”. Enzyme variants comprising conservative mutations appear to have a minimal effect on protein folding resulting in certain enzyme properties being substantially maintained when compared to the enzyme properties of the parent enzyme.

Conservative amino acid substitutions may occur over the full length of the sequence of a polypeptide sequence of a functional protein such as an enzyme. In one embodiment, such mutations are not pertaining the functional domains of an enzyme. In one embodiment, conservative mutations are not pertaining the catalytic centers of an enzyme.

For example, Amino acid A is similar to amino acids S; Amino acid D is similar to amino acids E; N; Amino acid E is similar to amino acids D; K; Q; Amino acid F is similar to amino acids W; Y; Amino acid H is similar to amino acids N; Y; Amino acid I is similar to amino acids L; M; V; Amino acid K is similar to amino acids E; Q; R; Amino acid L is similar to amino acids I; M; V; Amino acid M is similar to amino acids I; L; V; Amino acid N is similar to amino acids D; H; S; Amino acid Q is similar to amino acids E; K; R; Amino acid R is similar to amino acids K; Q; Amino acid S is similar to amino acids A; N; T; Amino acid T is similar to amino acids S; Amino acid V is similar to amino acids I; L; M; Amino acid W is similar to amino acids F; Y; and Amino acid Y is similar to amino acids F; H; W.

Especially, variant enzymes comprising conservative mutations which are at least m % similar to the respective parent sequences with m being an integer between 50 and 100, preferably 50, 55, 60, 65, 70, 75, 80, 85, 90, 91, 92, 93, 94, 95, 96, 97, 98 or 99 compared to the full-length polypeptide sequence, are expected to have essentially unchanged enzyme properties. Preferably, variant enzymes with m %-similarity when compared to a parent enzyme, have enzymatic activity.

Homologous refers to a gene, polypeptide, polynucleotide with a high degree of similarity, e.g. in position, structure, function or characteristic, but not necessarily with a high degree of sequence identity.

As used herein, “substantially complementary or substantially matched” means that two nucleic acid sequences have at least about 90% sequence identity. Preferably, the two nucleic acid sequences have at least, or at least about, 95%, 96%, 97%, 98%, 99%, or 100% of sequence identity. Alternatively, “substantially complementary or substantially matched” means that two nucleic acid sequences can hybridize under high stringency condition(s).

The term “hybridization” as defined herein is a process wherein substantially complementary nucleotide sequences anneal to each other. The hybridization process can occur entirely in solution, i.e. both complementary nucleic acids are in solution. The hybridization process can also occur with one of the complementary nucleic acids immobilized to a matrix such as magnetic beads, Sepharose beads or any other resin. The hybridization process can furthermore occur with one of the complementary nucleic acids immobilized to a solid support such as a nitro-cellulose or nylon membrane or immobilized by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids. Hybridization according to this description means, that hybridization must occur over complete length of the sequence of the invention. Such hybridization over the complete length, as defined herein, means, that when the sequence herein is fragmented into pieces of 300-500 bases, each fragment will hybridized.

The term “stringency” refers to the conditions under which hybridization takes place. The stringency of hybridization is influenced by conditions such as temperature, salt concentration, ionic strength and hybridization buffer composition. Generally, low stringency conditions are selected to be about 30° C. lower than the thermal melting point (Tm) for the specific sequence at a defined ionic strength and ph. Medium stringency conditions are when the temperature is 20° C. below Tm, and high stringency conditions are when the temperature is 10° C. below Tm. High stringency hybridization conditions are typically used for isolating hybridizing sequences that have high sequence identity to the target nucleic acid sequence. However, nucleic acids may deviate in sequence and still encode a substantially identical polypeptide, due to the degeneracy of the genetic code. Therefore, medium stringency hybridization conditions may sometimes be needed to identify such nucleic acid molecules. The “Tm” is the temperature under defined ionic strength and pH, at which 50% of the target sequence hybridizes to a perfectly matched probe. The Tm is dependent upon the solution conditions and the base composition and length of the probe. For example, longer sequences hybridize specifically at higher temperatures. The maximum rate of hybridization is obtained from about 16° C. up to 32° C. below Tm. The presence of monovalent cations in the hybridization solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridization to be performed at 30 to 45° C., though the rate of hybridisation will be lowered. Base pair mismatches reduce the hybridization rate and the thermal stability of the duplexes. On average and for large probes, the Tm decreases about 1° C. per % base mismatch. The Tm may be calculated according to routine knowledge in the art. Besides the hybridization conditions, specificity of hybridization typically also depends on the function of post-hybridization washes. The skilled artisan is aware of various parameters which may be altered during washing and which will either maintain or change the stringency conditions.

For example, typical high stringency hybridization conditions for DNA hybrids longer than 50 nucleotides encompass hybridization at 65° C. in 1×SSC or at 42° C. in 1×SSC and 50% formamide, followed by washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

As used herein, a “primer” refers to a nucleic acid molecule that can anneal to a template nucleic acid and serves as a starting point for DNA amplification. The primer can be entirely or partially complementary to a specific region of the template polynucleotide, for example 20 nucleotides upstream or downstream from a codon of interest. A non complementary nucleotide is defined herein as a mismatch. A mismatch may be located within the primer or at the either end of the primer. Preferably, a single nucleotide mismatch, more preferably two, and more preferably, three or more consecutive or not consecutive nucleotide mismatches is (are) located within the primer. The primer can have, for example, from 5 to 200 nucleotides, preferably, from 20 to 80 nucleotides, and more preferably, from 43 to 65 nucleotides. More preferably, the primer has 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, or 190 nucleotides. A “forward primer” as defined herein is a primer that is complementary to a minus strand of the template polynucleotide. A “reverse primer” as defined herein is a primer complementary to a plus strand of the template polynucleotide. Preferably, the forward and reverse primers do not comprise overlapping nucleotide sequences. “Do not comprise overlapping nucleotide sequences” as defined herein means that a forward and reverse primer does not anneal to a region of the minus and plus strands, respectively, of the template polynucleotide in which the plus and minus strands are complimentary to one another. With regard to the primers annealing to the same strand of the template polynucleotide, “do not comprise overlapping nucleotide sequences” means the primers do not comprise sequences complementary to the same region of the same strand of the template polynucleotide. As used herein, a “primer set” refers to a combination of a “forward primer” and a corresponding “reverse primer.”

As used herein, the plus strand equivalent to the sense strand and may also be referred to as a coding or non-template strand. This is the strand that has the same sequence as the mRNA (except it has Ts instead of Us). The other strand, called the template, minus, or antisense strand, is complementary to the mRNA.

As described herein, “codon optimization” refers to the design process of altering codons to codons known to increase maximum protein expression efficiency. In some alternatives, codon optimization for expression in a cell is described, wherein codon optimization can be performed by using algorithms that are known to those skilled in the art so as to create synthetic genetic transcripts optimized for high mRNA and protein yield in a host cell of interest, for example bacterial, fungal, insect, or mammalian cells (including human cells). Codons can be optimized for protein expression in a bacterial cell, mammalian cell, yeast cell, insect cell, or plant cell, for example. Programs containing algorithms for codon optimization in human cells are readily available. In some embodiments, the genes are codon optimized for expression in bacterial, yeast, fungal or insect cells.

The term “heterologous” (or exogenous or foreign or recombinant) polypeptide is defined herein as: (a) a polypeptide that is not native to the host cell. The protein sequence of such a heterologous polypeptide is a synthetic, non-naturally occurring, “man made” protein sequence; (b) a polypeptide native to the host cell in which structural modifications, e.g., deletions, substitutions, and/or insertions, have been made to alter the native polypeptide; or (c) a polypeptide native to the host cell whose expression is quantitatively altered or whose expression is directed from a genomic location different from the native host cell as a result of manipulation of the DNA of the host cell by recombinant DNA techniques, e.g., a stronger promoter.

Descriptions b) and c), above, refer to a sequence in its natural form but not naturally expressed by the cell used for its production. The produced polypeptide is therefore more precisely defined as a “recombinantly expressed endogenous polypeptide”, which is not in contradiction to the above definition but reflects the specific situation that it's not the sequence of a protein being synthetic or manipulated but the way the polypeptide molecule is produced.

Similarly, the term “heterologous” (or exogenous or foreign or recombinant) polynucleotide refers: (a) to a polynucleotide that is not native to the host cell; (b) a polynucleotide native to the host cell in which structural modifications, e.g., deletions, substitutions, and/or insertions, have been made to alter the native polynucleotide; (c) a polynucleotide native to the host cell whose expression is quantitatively altered as a result of manipulation of the regulatory elements of the polynucleotide by recombinant DNA techniques, e.g., a stronger promoter; or (d) a polynucleotide native to the host cell, but integrated not within its natural genetic environment as a result of genetic manipulation by recombinant DNA techniques.

With respect to two or more polynucleotide sequences or two or more amino acid sequences, the term “heterologous” is used to characterize that the two or more polynucleotide sequences or two or more amino acid sequences do not occur naturally in the specific combination with each other.

As used herein, “transgenic”, “transgene” or “recombinant” means with regard to, for example, a nucleic acid sequence, an expression cassette, genetic construct or a vector comprising the nucleic acid sequence or an organism transformed with the nucleic acid sequences, expression cassettes or vectors, all those constructions brought about synthetically by recombinant or gene-technological methods in which either (a) the nucleic acid sequences comprising desired genetic information to be expressed, or (b) genetic control sequence(s) which is operably linked with the nucleic acid sequence comprising said desired genetic information, for example a promoter, or (c) both (a) and (b), are not located in their natural genetic environment or have been modified by recombinant methods. The natural genetic environment is understood as meaning the natural genomic or chromosomal locus in the original organism. A naturally occurring expression cassette—for example the naturally occurring combination of the natural promoter of the nucleic acid sequences with the corresponding nucleic acid sequence encoding a polypeptide, becomes a transgenic expression cassette when this expression cassette is modified through human intervention such as, for example, mutagenic treatment. Furthermore, a naturally occurring expression cassette becomes a recombinant expression cassette when this expression cassette is isolated from its natural genetic environment and subsequently reintroduced in a genetic environment that is not the natural genetic environment.

A “synthetic” or “artificial” compound is produced by in vitro chemical or enzymatic synthesis. It includes, but is not limited to, variant nucleic acids made with optimal codon usage for host organisms, such as a yeast cell host or other expression hosts of choice or variant protein sequences with amino acid modifications, such as e.g. substitutions, compared to the parent protein sequence, e.g. to optimize properties of the polypeptide.

A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length.

The terms “fragment”, “derivative” and “analog” when referring to a reference polypeptide comprise a polypeptide which retains at least one biological function or activity that is at least essentially same as that of the reference polypeptide.

The term “functional fragment” refers to any nucleic acid or amino acid sequence which comprises merely a part of the full length nucleic acid or full length amino acid sequence, respectively, but still has the same or similar activity and/or function. In one embodiment, the fragment comprises at least 70%, at least 80%, at least 85%, at least 90%, at least 95%, at least 96%, at least 97%, at least 98%, at least 99% of the original sequence. In one embodiment, the functional fragment comprises contiguous nucleic acids or amino acids compared to the original nucleic acid or original amino acid sequence, respectively.

The term “gene” means the segment of DNA involved in producing a polypeptide chain; it includes regions preceding and following the coding region (leader and trailer) as well as optionally intervening sequences (introns) between individual coding segments (exons).

As used herein, the term “isolated” means that the material is removed from its original environment (e.g., the natural environment if it is naturally occurring). For example, a naturally-occurring polynucleotide or enzyme present in a living animal is not isolated, but the same polynucleotide or enzyme, separated from some or all of the coexisting materials in the natural system, is isolated. Such polynucleotides could be part of a vector and/or such polynucleotides or enzymes could be part of a composition, and still be isolated in that such vector or composition is not part of its natural environment. As further example, an isolated nucleic acid, e.g., a DNA or RNA molecule, is one that is not immediately contiguous with the 5′ and 3′ flanking sequences with which it normally is immediately contiguous when present in the naturally occurring genome of the organism from which it is derived. Such polynucleotides could be part of a vector, incorporated into a genome of a cell with an unrelated genetic background (or into the genome of a cell with an essentially similar genetic background, but at a site different from that at which it naturally occurs), or produced by PCR amplification or restriction enzyme digestion, or an RNA molecule produced by in vitro transcription, and/or such polynucleotides, polypeptides, or enzymes could be part of a composition, and still be isolated in that such vector or composition is not part of its natural environment.

The term “isolated” means that the DNA is incorporated into a vector, such as a plasmid or viral vector; a nucleic acid that is incorporated into the genome of a heterologous cell (or the genome of a homologous cell, but at a non-naturally occurring site); and a nucleic acid that exists as a separate molecule, e.g., a DNA fragment produced by PCR amplification or restriction enzyme digestion, or an RNA molecule produced by in vitro transcription.

As used herein, the term “purified” does not require absolute purity; rather, it is intended as a relative definition. Individual nucleic acids obtained from a library have been conventionally purified to electrophoretic homogeneity. For example, the purified nucleic acids of the present disclosure can be purified from the remainder of the genomic DNA in the organism by at least 104-106 folds. However, the term “purified” also includes nucleic acids which have been purified from the remainder of the genomic DNA or from other sequences in a library or other environment by at least one order of magnitude, typically two or three orders, and more typically four or five orders of magnitude. “Purified” means that the material is in a relatively pure state, e.g., at least about 90% pure, at least about 95% pure, or at least about 98% or 99% pure. Preferably “purified” means that the material is in a 100% pure state.

The term “operably linked” means that the described components are in a relationship permitting them to function in their intended manner. For example, a regulatory sequence operably linked to a coding sequence is ligated in such a way that expression of the coding sequence is achieved under condition compatible with the control sequences. As used herein, a promoter sequence is “operably linked to” a coding sequence when RNA polymerase which initiates transcription at the promoter can transcribe the coding sequence into mRNA.

The term “mutations” is defined as alterations in the genetic code of nucleic acid sequence or alterations in the sequence of a peptide. Such mutations may be point mutations such as transitions or transversions. A mutation may be a change to one or more nucleotides or encoded amino acid sequences. The mutations may be deletions, insertions or duplications.

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

The terms “nucleic acid sequence coding for” or a “DNA coding sequence of” or a “nucleotide sequence encoding” a particular protein or polypeptide refer to a DNA sequence which is transcribed and translated into a protein or polypeptide when placed under the control of appropriate regulatory sequences.

The terms “nucleic acid encoding a protein or peptide” or “DNA encoding a protein or peptide” or “polynucleotide encoding a protein or peptide” and other synonymous terms encompasses a polynucleotide which includes only coding sequence for the protein or peptide as well as a polynucleotide which includes additional coding and/or non-coding sequence.

The terms “regulatory element”, “control sequence” and “promoter” are all used interchangeably herein and are to be taken in a broad context to refer to regulatory nucleic acid sequences capable of effecting expression of the sequences to which they are associated. “Regulatory elements” or “regulatory nucleotide sequences” herein may mean pieces of nucleic acid which drive expression of a nucleic acid sequence upon transformation into a host cell or cell organelle had occurred. Regulatory nucleotide sequences may include any nucleotide sequence having a function or purpose individually and within a particular arrangement or grouping of other elements or sequences within the arrangement. Examples of regulatory nucleotide sequences include but are not limited to transcription control elements such as promoters, enhancers, and termination elements. Regulatory nucleotide sequences may be native (i.e. from the same gene) or foreign (i.e. from a different gene) to a nucleotide sequence to be expressed.

The term “promoter” typically refers to a nucleic acid control sequence located upstream from the transcriptional start of a gene and is involved in recognizing and binding of RNA polymerase and other proteins, thereby directing transcription of an operably linked nucleic acid. “Promoter” herein may further include any nucleic acid sequence capable of driving transcription of a coding sequence. In particular, the term “promoter” as used herein may refer to a polynucleotide sequence generally described as the 5′ regulator region of a gene, located proximal to the start codon. The transcription of one or more coding sequence is initiated at the promoter region. The term promoter may also include fragments of a promoter that are functional in initiating transcription of the gene. Promoter may also be called “transcription start site” (TSS).

Encompassed by the aforementioned terms are further transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner.

For example, enhancers as known in the art and as used herein are normally short DNA segments (e.g. 50-1500 bp) which may be bound by proteins such as transcription factors to increase the likelihood that transcription of a coding sequence will occur.

Further elements may be “transcription termination elements” which include pieces of nucleic acid sequences marking the end of a gene and mediating the transcriptional termination by providing signals within mRNA that initiates the release of the mRNA from the transcriptional complex. Transcriptional termination in prokaryotes and eukaryotes are conventional knowledge in the art.

An “oligonucleotide” (or synonymously an “oligo”) refers to either a single stranded poly-deoxynucleotide or two complementary poly-deoxynucleotide strands which may be chemically synthesized. Such synthetic oligonucleotides may or may not have a 5′ phosphate.

Any source of nucleic acid, in purified form can be utilized as the starting nucleic acid (also defined as “a template polynucleotide”). Thus, the process may employ DNA or RNA including messenger RNA, which DNA or RNA can be single-stranded, and preferably double stranded. In addition, a DNA-RNA hybrid which contains one strand of each may be utilized. The nucleic acid sequence may be of various lengths depending on the size of the nucleic acid sequence to be mutated. Preferably the specific nucleic acid sequence is from 50 to 50000 base pairs, and more preferably from 50-11000 base pairs.

All methods and materials similar or equivalent to those described herein can be used in the practice or testing of methods and compositions disclosed herein, with suitable methods and materials being described herein. All publications, patent applications, patents, and other references mentioned herein are incorporated by reference in their entirety. Further, the materials, methods, and examples are illustrative only and are not intended to be limiting, unless otherwise specified.

### Nuclease Polypeptides

Disclosed herein are polypeptides having nuclease activity and salt-tolerant. To overcome the defect of the nuclease under high salt concentration, the inventor engineered the nuclease from Serratia marcescens by introducing positive amino acid residues to reshape its electrostatics surface. After screening, a nuclease mutant was able to tolerant at least 200 mM NaCl, without significant enzyme activity decrease.

This mutant is able to expand its usage to higher salt solutions.

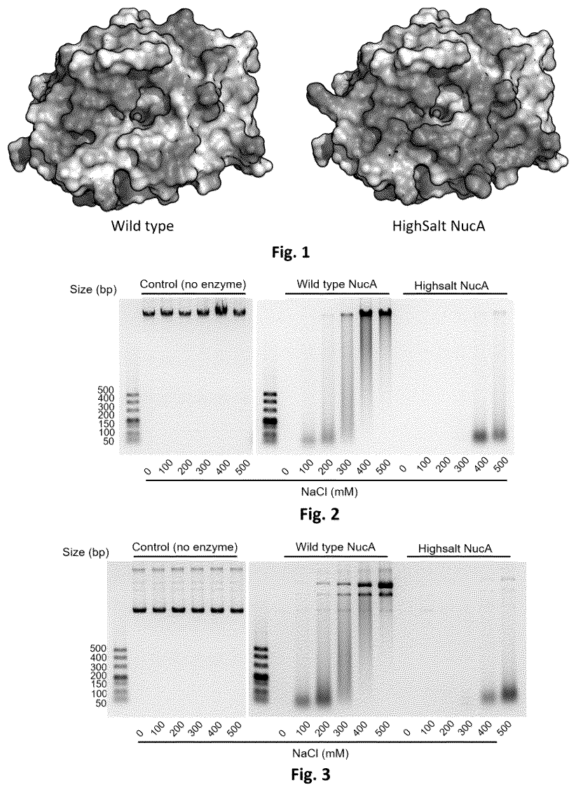

Serratia marcescens nuclease A is an extracellular enzyme and has been widely used to remove nucleic acids for numerous applications. However, this enzyme is salt-sensitive, and its enzyme activity is greatly inhibited by >200 mM solution ion strength. By analyzing the crystal structure of nuclease A, the interactions between nuclease A and nucleic acids are basically dependent on electrostatic interaction. High salt concentration abolishes those interactions, which leads to inhibition of enzyme activity of nuclease A by high salt condition. Thus, enhance nucleic acids-protein interactions is an effective way to improve nuclease A's salt tolerance. Based on this principle, the inventor engineered Serratia marcescens nuclease A by introducing more positive charged residues to the predicted nucleic acid binding surface, in which residues S74, N79, A95, T98, N101, A102, S137, D138, Q141, D149, D156 are included. Mutation of these residues to certain amino acid will make the nucleic acids binding surface more positive, and enhance the binding of nucleic acids. It should be noticed that improved nucleic acid binding may also affect the dissociation of cleaved nucleic acid from protein. The enzyme activity may also be inhibited by too strong nucleic acid binding. Thus, a balance between nucleic acid affinity and enzyme activity should be compromised. The inventor designed 6 mutants by combining of mutations from above 11 residues, and all mutants were expressed and purified. By comparing the electrostatic potential surface of wild type nuclease A and the mutated nuclease A, the inventor found that mutated nuclease A indeed possesses an enlarged positive charged surface (FIG. 1). Rational engineering Serratia marcescens nuclease A by introducing positive charged residues to specific regions of its surface significantly enhances the enzyme tolerance towards high salt condition.

In some embodiments, the mutation of N79 is a mutation to a non polar amino acids such as alanine, valine, isoleucine, proline, phenylalanine, tryptophan methionine, or glycine, or to an uncharged polar amino acid such as serine, threonine, glutamine, tyrosine, or cysteine, or to a polar amino acid with positive charges such as histidine, lysine or arginine. In certain embodiments, the mutation of N79 is a mutation to Ala, Cys, Gly, Gln, Ser, Thr, Val, Trp, Tyr, Lys, or Arg.

In some embodiments, the mutation of A95 is a mutation to a non polar amino acids such as valine, isoleucine, proline, phenylalanine, tryptophan methionine, or glycine, or to an uncharged polar amino acid such as serine, threonine, glutamine, asparagine, tyrosine, or cysteine, or to a polar amino acid with positive charges such as histidine, lysine or arginine. In certain embodiments, the mutation of A95 is a mutation to Phe, Asn, Pro, Gln, Ser, Thr, Val, Tyr, Lys, or Arg.

In some embodiments, the mutation of A102 is a mutation to a non polar amino acids such as valine, isoleucine, proline, phenylalanine, tryptophan methionine, or glycine, or to an uncharged polar amino acid such as serine, threonine, glutamine, asparagine, tyrosine, or cysteine, or to a polar amino acid with positive charges such as histidine, lysine or arginine. In certain embodiments, the mutation of A102 is a mutation to Thr, Val, Trp, Tyr, Lys, or Arg.

In some embodiments, the mutation of D149 is a mutation to a non polar amino acids such as alanine, valine, isoleucine, proline, phenylalanine, tryptophan methionine, or glycine, or to an uncharged polar amino acid such as serine, threonine, glutamine, asparagine, tyrosine, or cysteine, or to a polar amino acid with positive charges such as histidine, lysine or arginine. In certain embodiments, the mutation of D149 is a mutation to Ala, Phe, His, Asn, Gln, Ser, Thr, Val, Trp, Tyr, Lys, or Arg.

In some embodiments, the mutation of S74, N79, A95, T98, N101, A102, S137, D138, Q141, D149, D156 is a mutation to a polar amino acid with positive charges, preferably histidine, lysine or arginine, more preferably lysine or arginine.

Serratia marcescens nuclease A is a polypeptide having the amino acid sequence of SEQ ID NO: 1 which exhibits nuclease activity. The first 21 amino acids on the N-terminus of SEQ ID NO: 1 is the signal sequence, and the remaining amino acids form the mature polypeptide.

In some embodiments, the polypeptide disclosed herein is an isolated, synthetic, or recombinant polypeptide comprising an amino acid sequence having at least 80%, 85%, 90%, 95%, 98%, 99%, or more sequence identity to SEQ ID NO: 1 or the mature polypeptide thereof, where the polypeptide has nuclease activity. The polypeptide can, for example, has 80%, 85%, 90%, 91%, 92%, 93%, 94%, 95%, 96%, 97%, 98%, 99%, 99.5%, or a range between any two of these values, sequence identity to SEQ ID NO: 1 or the mature polypeptide thereof.

In some embodiments, the polypeptide: (a) comprises mutations at 1, 2, 3, 4, 5, 6, 7 or more or all sites of S74, N79, A95, T98, N101, A102, S137, D138, Q141, D149, D156 of SEQ ID NO:1. The mutations include modification, substitution or deletion of amino acids. In some embodiments, a polypeptide comprising an amino acid sequence having at least 70% sequence identity to the sequence of (a) and having the mutation at corresponding site as described in (a) retains nuclease activity. The amino acid mutations are described herein relative to the corresponding amino acid position in SEQ ID NO: 1. For example, an amino acid substitution from S to K at position 74 of SEQ ID NO: 1 is described herein as S74K.

In some embodiments, the polypeptide has mutations at at least 4 sites selected from the group consisting of S74, N79, A95, T98, N101, A102, S137, D138, Q141, D149, D156 of SEQ ID NO: 1 or the mature polypeptide thereof. In some embodiments, the polypeptide has a mutation at D149 and at least 3 sites selected from the group consisting of S74, N79, A95, T98, N101, A102, S137, D138, Q141, D156 of SEQ ID NO:1. In certain embodiments, the mutation of each site is a mutation to lysine or arginine.

In some embodiments, the polypeptide comprises an amino acid sequence having at least 70% identity to SEQ ID NO: 1 or the mature polypeptide thereof, and has mutations at at least three sites selected from the group consisting of N79, A95, A102, and D149 and optionally at least one site selected from the group consisting of S74, T98, N101, S137, D138, Q141, D156. In certain embodiments, the mutation of each site is a mutation to lysine or arginine.

In some embodiments, the polypeptide comprises an amino acid sequence having at least 70% identity to SEQ ID NO: 1 or the mature polypeptide thereof and has a mutation at A95, A102, and D149 and optionally at least one sites selected from the group consisting of S74, N79, T98, N101, S137, D138, Q141, D156. In certain embodiments, the mutation of each site is a mutation to lysine or arginine.

In some embodiments, the polypeptide comprises an amino acid sequence having at least 70% identity to SEQ ID NO: 1 or the mature polypeptide thereof and has a mutation at N79, A102, and D149 and optionally at least one sites selected from the group consisting of S74, A95, T98, N101, S137, D138, Q141, D156. In certain embodiments, the mutation of each site is a mutation to lysine or arginine.

In some embodiments, the polypeptide comprises an amino acid sequence having at least 70% identity to SEQ ID NO: 1 or the mature polypeptide thereof and has a mutation at N79, A95, and D149 and optionally at least one sites selected from the group consisting of S74, T98, N101, A102, S137, D138, Q141, D156. In certain embodiments, the mutation of each site is a mutation to lysine or arginine.

In some embodiments, the polypeptide comprises an amino acid sequence having at least 70% identity to SEQ ID NO: 1 or the mature polypeptide thereof and has a mutation at N79, A95, and A102 and optionally at least one sites selected from the group consisting of S74, T98, N101, A102, S137, D138, Q141, D149, D156. In certain embodiments, the mutation of each site is a mutation to lysine or arginine.

In some embodiments, the polypeptide has mutations at N79, A95, A102, and D149 and optionally at least one site selected from the group consisting of S74, T98, N101, S137, D138, Q141, D156 of SEQ ID NO:1. In some embodiments, the polypeptide has mutations at N79, A95, A102, and D149 and optionally at one or two sites selected from the group consisting of Q141 and N101. In certain embodiments, the mutation of each site is a mutation to lysine or arginine.

In some embodiments, the polypeptide has mutations at A95, N101, Q141, D149 of SEQ ID NO:1 and optionally at least 3 sites selected from the group consisting of S74, N79, T98, A102, S137, D138, D156. In some embodiments, the polypeptide has mutations at A95, N101, Q141, D149 and optionally at S74, N79, and T98. In certain embodiments, the mutation of each site is a mutation to lysine or arginine.

In some embodiments, the polypeptide can be a polypeptide comprising an amino acid sequence having at least 70%, at least 80%, at least 85%, at least 90%, at least 95%, or at least 99% sequence identity to SEQ ID NO: 1 or the mature polypeptide thereof, where the polypeptide has nuclease activity under high solution ionic strength, and where the polypeptide comprises one or more mutations of (1) N79K or N79R, (2) A95K or A95R, (3) A102K or A102R, (4) D149K or D149R, (5) S74K, (6) T98K, (7) N101K, (8) S137K, (9) D138K, (10) Q141K, and (11) D156R; for example, one or more mutations of S74K, N79K, A95K, T98K, N101K, A102K, S137K, D138K, Q141K, D149K, and D156R.

In some embodiments, the polypeptide has at least 4 mutations selected from the group consisting of (1) N79K or N79R, (2) A95K or A95R, (3) A102K or A102R, (4) D149K or D149R, (5) S74K, (6) T98K, (7) N101K, (8) S137K, (9) D138K, (10) Q141K, and (11) D156R, (for example, the group consisting of S74K, N79K, A95K, T98K, N101K, A102K, S137K, D138K, Q141K, D149K, and D156R) in SEQ ID NO: 1. In some embodiments, the polypeptide has D149K or D149R mutation and at least 3 (or at least 4, at least 5, at least 6, at least 7 or more) mutations selected from the group consisting of (1) N79K or N79R, (2) A95K or A95R, (3) A102K or A102R, (5) S74K, (6) T98K, (7) N101K, (8) S137K, (9) D138K, (10) Q141K, and (11) D156R, (for example, the group consisting of S74K, N79K, A95K, T98K, N101K, A102K, S137K, D138K, Q141K, and D156R) in SEQ ID NO:1.

In some embodiments, the polypeptide comprises an amino acid sequence having at least 70% identity to SEQ ID NO: 1 or the mature polypeptide thereof and has at least three mutations selected from the group consisting of (1) N79K or N79R, (2) A95K or A95R, (3) A102K or A102R, (4) D149K or D149R, and optionally has a mutation selected from the group consisting of S74K, T98K, N101K, S137K, D138K, Q141K, and D156R.

In some embodiments, the polypeptide comprises an amino acid sequence having at least 70% identity to SEQ ID NO: 1 or the mature polypeptide thereof and has three mutations of (1) N79K or N79R, (2) A95K or A95R, and (3) A102K or A102R, and optionally has a mutation selected from the group consisting of (4) D149K or D149R, (5) S74K, (6) T98K, (7) N101K, (8) S137K, (9) D138K, (10) Q141K, and (11) D156R.

In some embodiments, the polypeptide comprises an amino acid sequence having at least 70% identity to SEQ ID NO: 1 or the mature polypeptide thereof and has three mutations of (1) N79K or N79R, (2) A95K or A95R, and (3) D149K or D149R, and optionally has a mutation selected from the group consisting of (4) A102K or A102R, (5) S74K, (6) T98K, (7) N101K, (8) S137K, (9) D138K, (10) Q141K, and (11) D156R.

In some embodiments, the polypeptide comprises an amino acid sequence having at least 70% identity to SEQ ID NO: 1 or the mature polypeptide thereof and has three mutations of (1) N79K or N79R, (2) A102K or A102R and (3) D149K or D149R, and optionally has a mutation selected from the group consisting of (4) A95K or A95R, (5) S74K, (6) T98K, (7) N101K, (8) S137K, (9) D138K, (10) Q141K, and (11) D156R.

In some embodiments, the polypeptide comprises an amino acid sequence having at least 70% identity to SEQ ID NO: 1 or the mature polypeptide thereof and has three mutations of (1) A95K or A95R, (2) A102K or A102R and (3) D149K or D149R, and optionally has a mutation selected from the group consisting of (4) N79K or N79R, (5) S74K, (6) T98K, (7) N101K, (8) S137K, (9) D138K, (10) Q141K, and (11) D156R.

In some embodiments, the polypeptide comprises an amino acid sequence having at least 70% identity to SEQ ID NO: 1 or the mature polypeptide thereof and has three mutations of (1) A95K or A95R, (2) A102K or A102R and (3) D149K or D149R, and optionally has a mutation selected from the group consisting of (4) N79K or N79R, (5) S74K, (6) T98K, (7) N101K, (8) S137K, (9) D138K, (10) Q141K, and (11) D156R.

In some embodiments, the polypeptide comprises an amino acid sequence having at least 70% identity to SEQ ID NO: 1 or the mature polypeptide thereof and has three mutations of (i) A95K, A102K, and D149K, or (ii) A95R, A102R, and D149R, and optionally has a mutation selected from the group consisting of (1) N79K or N79R, (2) S74K, (3) T98K, (4) N101K, (5) S137K, (6) D138K, (7) Q141K, and (8) D156R.

In some embodiments, the polypeptide comprises an amino acid sequence having at least 70% identity to SEQ ID NO: 1 or the mature polypeptide thereof and has three mutations of (i) N79K, A102K, and D149K, or (ii) N79R, A102R, and D149R, and optionally has a mutation selected from the group consisting of (1) A95K or A95R, (2) S74K, (3) T98K, (4) N101K, (5) S137K, (6) D138K, (7) Q141K, and (8) D156R.

In some embodiments, the polypeptide comprises an amino acid sequence having at least 70% identity to SEQ ID NO: 1 or the mature polypeptide thereof and has three mutations of (i) N79K, A95K, and D149K, or (ii) N79R, A95R, and D149R, and optionally has a mutation selected from the group consisting of (1) A102K or A102R, (2) S74K, (3) T98K, (4) N101K, (5) S137K, (6) D138K, (7) Q141K, and (8) D156R.

In some embodiments, the polypeptide comprises an amino acid sequence having at least 70% identity to SEQ ID NO: 1 or the mature polypeptide thereof and has three mutations of (i) N79K, A95K, and A102K or (ii) N79R, A95R, and A102R, and optionally has a mutation selected from the group consisting of (1) D149K or D149R, (2) S74K, (3) T98K, (4) N101K, (5) S137K, (6) D138K, (7) Q141K, and (8) D156R.

In some embodiments, the polypeptide comprises an amino acid sequence having at least 70% identity to SEQ ID NO: 1 or the mature polypeptide thereof and has three mutations of (i) N79K, A95K, A102K, and D149K or (ii) N79R, A95R, A102R and D149R, and optionally has a mutation selected from the group consisting of (1) S74K, (2) T98K, (3) N101K, (4) S137K, (5) D138K, (6) Q141K, and (7) D156R.

In some embodiments, the polypeptide has mutations of (i) N79K, A95K, A102K, D149K, or (ii) N79R, A95R, A102R and D149R, and optionally has a mutation selected from the group consisting of S74K, T98K, N101K, S137K, D138K, Q141K, and D156R. In some embodiments, the polypeptide has mutations of (i) N79K, A95K, A102K, and D149K or (ii) N79R, A95R, A102R and D149R and optionally has a mutation selected from the group consisting of Q141K and N101K.

In some embodiments, the polypeptide has mutations of A95K, N101K, Q141K, D149K in SEQ ID NO:1 and optionally has a mutation selected from the group consisting of S74K, N79K, T98K, A102K, S137K, D138K, and D156R. In some embodiments, the polypeptide has mutations of A95K, N101K, Q141K, D149K and optionally mutations of S74K, N79K, and T98K. In some embodiments, the polypeptide has mutations of S74K, N79K, A95K, T98K, N101K, A102K, S137K, D138K, Q141K, D149K, and D156R in SEQ ID NO:1.

In some embodiments, the polypeptide comprises a mutation or a combination of mutations selected from the group consisting of: (a) N79K, A95K, A102K, and D149K; (b) N79K, A95K, A102K, Q141K, and D149K; (c) N79K, A95K, N101K, A102K, Q141K, and D149K; (d) A95K, N101K, Q141K, and D149K; (e) S74K, N79K, A95K, T98K, N101K, Q141K, and D149K; (f) S74K, N79K, A95K, T98K, N101K, A102K, S137K, D138K, Q141K, D149K, and D156R.

In some embodiments, the polypeptide is any one of the nuclease variants disclosed herein, where the polypeptide has nuclease activity.

In some embodiments, the nuclease polypeptides are thermos-tolerant. For example, the nuclease polypeptide can be more thermos-tolerant than the nuclease having the sequence of SEQ ID NO: 1. In some embodiments, the nuclease activity of the polypeptide is at least 1%, 2%, 3%, 4%, 5%, 7%, 10%, 15%, 20%, 25%, 30%, 35%, 40%, 45%, 50%, or more higher than that of the nuclease having the sequence of SEQ ID NO: 1 at a given temperature, for example at a temperature between 30° C. and 60° C. In some embodiments, the nuclease activity of the polypeptide is 1%, 2%, 3%, 4%, 5%, 7%, 10%, 15%, 20%, 25%, 30%, 35%, 40%, 45%, 50%, 75%, 100%, or a range between two of these values, higher than that of the nuclease having the sequence of SEQ ID NO: 1 at a given temperature, for example at a temperature between 30° C. and 60° C. In some embodiments, the nuclease activity of the polypeptide is at least 1%, 2%, 3%, 4%, 5%, 7%, 10%, 15%, 20%, 25%, 30%, 35%, 40%, 45%, 50%, or more higher than that of the nuclease having the sequence of SEQ ID NO: 1 at a given pH, for example at pH 4 to pH 11.

In some embodiments, the nuclease polypeptides are salt-tolerant. In some embodiments, the polypeptide with mutations has at least 60%, at least 70%, at least 80%, at least 90%, or at least 95% nuclease activity under a solution ionic strength more than 200 mM as compared to the nuclease having the sequence of SEQ ID NO: 1. The ion herein is a monovalent ion or divalent ion. In some embodiments, the solution ionic strength is more than 300 mM, for example more than 400 mM, 500 mM, 600 mM, 700 mM, 800 mM, 900 mM, 1 M, 2 M, 3 M, 4 M, 5 M or a higher ionic strength.

The optimal ionic strength of the polypeptide can be different (for example higher or lower) than that of the nuclease having the sequence of SEQ ID NO: 1 or its parent nuclease. For example, the optimal ionic strength of the polypeptide can be 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, or a range between any two of these values, higher than the optimal ionic strength of the nuclease having the sequence of SEQ ID NO: 1. In some embodiments, the optimal ionic strength of the polypeptide is, or is about, 50 mM, 60 mM, 70 mM, 80 mM, 90 mM, 100 mM, 200 mM, 300 mM, 400 mM, 500 mM, 600 mM, 700 mM, 800 mM, 900 mM, or a range between any two of these values. In some embodiments, the optimal temperature of the polypeptide is between 100 mM and 600 mM.

During the fermentation process, DNA from a production host can complicate many aspects of the protein recovery process. Currently, DNase has been added in order to remove the DNA from final product, which often requires addition of costly materials from other sources. The nucleases disclosed herein can be expressed, in some embodiments, in the same production host as product of interest, which eliminates the needs to add external DNase so that can reduce the overall cost of processing to final product.

The nuclease polypeptides disclosed herein can have one or more signal sequences. In some embodiments, at least one of the one or more signal sequences is heterologous to the nuclease polypeptide it is comprised in. In some embodiments, the nuclease polypeptides disclosed herein do not contain any signal sequences.

Also disclosed herein are antibody or binding fragment thereof (e.g., isolated or purified antibody or binding fragment thereof) which specifically binds to an isolated, synthetic, or recombinant polypeptide comprising an amino acid sequence having at least 80%, 85%, 90%, 95%, 98%, 99%, or more sequence identity to SEQ ID NO: 1 or the mature polypeptide thereof, where the polypeptide has nuclease activity. In some embodiments, the polypeptide comprises one or more mutations of S74K, N79K, A95K, T98K, N101K, A102K, S137K, D138K, Q141K, D149K, and D156R.

In some embodiments, the polypeptide comprises a combination of mutations selected from the group consisting of: (1) N79K, A95K, A102K, and D149K; (2) N79K, A95K, A102K, Q141K, and D149K; (3) N79K, A95K, N101K, A102K, Q141K, and D149K; (4) A95K, N101K, Q141K, and D149K; (5) S74K, N79K, A95K, T98K, N101K, Q141K, and D149K; (6) S74K, N79K, A95K, T98K, N101K, A102K, S137K, D138K, Q141K, D149K, and D156R. (7) N79R, A95R, A102R, D149R; (8) A95K, A102K, D149K; (9) N79K, A102K, D149K; (10) N79K, A95K, D149K; (11) N79K, A95K, A102K; (12) A95R, A102R, D149R; (13) N79R, A102R, D149R; (14) N79R, A95R, D149R; (15) N79R, A95R, A102R.

Variants of the nucleases disclosed herein can comprise one or more of substitutions, deletions, and insertions at one or more of the amino acid positions of the nucleases. In some embodiments, the number of amino acid substitutions, deletions and/or insertions introduced into the parent nuclease (for example the nuclease having the sequence of SEQ ID NO: 1) is not more than 30, for example, 1, 2, 3, 4, 5, 6, 7, 8, 9, 10, 11, 12, 13, 14, 15, 16, 17, 18, 19, 20, 21, 22, 23, 24, 25, 26, 27, 28, or 29. The amino acid changes can be of a minor nature, that is conservative amino acid substitutions or insertions that do not significantly affect the folding and/or activity of the protein; small deletions (for example 1-20 amino acids); small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain. In some embodiments, the amino acid changes to the nucleases can alter one or more physico-chemical properties of the parent nucleases.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Non-limiting exemplary amino acid substitutions include Ala to Ser, Val to Ile, Asp to Glu, Thr to Ser, Ala to Gly, Ala to Thr, Ser to Asn, Ala to Val, Ser to Gly, Tyr to Phe, Ala to Pro, Lys to Arg, Asp to Asn, Leu to Ile, Leu to Val, Ala to Glu, and Asp to Gly.

Amino acid substitutions, deletions, and/or insertions can be made and tested using methods known in the art for protein/DNA engineering, including but not limited to mutagenesis, recombination, and/or shuffling, followed by a relevant screening procedure. In some embodiments, mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide.

In some embodiments, the nuclease polypeptides can tolerate higher or lower ionic strengthen than other nucleases, for example the nuclease having the sequence of SEQ ID NO: 1. For example, the nuclease polypeptides can retain a nuclease activity (for example, at least 40%, 50%, 60%, 70%, 80%, 90%, 95%, or 98% of its nuclease activity) at, or at about, 50 mM, 60 mM, 70 mM, 80 mM, 90 mM, 100 mM, 200 mM, 300 mM, 400 mM, 500 mM, 600 mM, 700 mM, 800 mM, 900 mM, or a range between any two of these values.

The nuclease polypeptides disclosed herein can have the same or different substrate specificity as compared to the nuclease having the sequence of SEQ ID NO: 1 or its respective parent nuclease. For example, the nuclease polypeptides can have substantially the same substrate specificity as compared to the nuclease having the sequence of SEQ ID NO: 1 or its respective parent nuclease. In some embodiments, the nuclease polypeptides have about 50%, 60%, 70%, 80%, 90%, 95%, 98%, or a range between any two of these values, substrate specificity as compared to the nuclease having the sequence of SEQ ID NO: 1 or its respective parent nuclease. Also disclosed herein is a composition that comprises one or more of the nuclease polypeptides disclosed herein.

Also provided herein are immobilized nuclease polypeptides, wherein the immobilized polypeptide comprises one of the nuclease polypeptides disclosed herein. In some embodiments, the polypeptide can be immobilized on a cell, a metal, a resin, a polymer, a ceramic, a glass, a microelectrode, a graphitic particle, a bead, a gel, a plate, an array, a capillary tube, or a combination thereof.

### Nucleic Acid

The terms “nucleic acid” or “nucleotide” or “polynucleotide” or “nucleic acid sequence” used herein are interchangeable, and may be in the form of DNA or RNA. DNA includes cDNA, genomic DNA, or artificially synthesized DNA. DNA can be single stranded or double stranded. DNA can be either the coding or noncoding strand. When referring to nucleic acids, the term “variant” used herein may be a naturally occurring allelic variant or a non-naturally occurring variant. These nucleotide variants include degenerate variants, substitution variants, deletion variants, and insertion variants. As is known in the art, an allelic variant is a form of substitution of a nucleic acid, which may be a substitution, deletion, or insertion of one or more nucleotides but does not substantially alter the function of the protein it encodes. The nucleic acid of the invention may comprise a nucleotide sequence having sequence identity of at least about 70%, at least about 75%, at least about 80%, at least about 85%, at least about 90%, at least about 95%, at least about 98%, at least about 99%, or 100% to the nucleic acid sequence. The invention also relates to a nucleic acid fragment that hybridizes to the sequences described above. As used herein, a “nucleic acid fragment” is at least 15 nucleotides in length, preferably at least 30 nucleotides, more preferably at least 50 nucleotides, and even more preferably at least 100 nucleotides or more. The nucleic acid fragment can be used in amplification techniques of nucleic acids (such as PCR).

The full-length coding sequences or fragments of polypeptides of the invention can be obtained by PCR amplification, artificial synthesis or recombinant procedures. Mutations of the polypeptides can be introduced during PCR, synthesis or recombination.

For PCR amplification, primers can be designed according to the nucleotide sequences disclosed in the invention and the sequences in interest can be amplified with a commercially available cDNA library or a cDNA library prepared by conventional methods known to those skilled in the art as template. When the nucleotide sequence is greater than 2500 bp, it is preferable to perform 2~ 6 rounds of PCR amplification, and then the individually amplified fragments are spliced together in the correct order. There is no special limit to the procedures and systems for PCR amplification described herein, and conventional PCR amplification procedures and systems in the art may be used.

In addition, methods are also available to artificially synthesize the sequence in interest, especially for short fragments. For example, when the nucleotide sequence of the optical probe is less than 2500 bp, it may be synthesized by an artificial synthetic method. The artificial synthetic method can be any conventional artificial synthetic method for DNA in the art. In general, many small fragments are first synthesized, and then ligated to obtain a longer sequence. It is also possible to obtain, entirely by chemical synthesis, DNA sequences encoding the proteins of the invention. This DNA sequence can then be introduced into a variety of existing DNA molecules known in the art, such as vectors, and into cells.

Recombination can also be used to obtain relevant sequences in bulk. This is usually by cloning into a vector, retransferring into cells, and then isolating and purifying the polypeptide or protein in interest from proliferated host cells by conventional methods.

### Production of Nuclease Polypeptides and Variants Thereof

Provided herein are methods for modifying and making variants of nuclease polynucleotides disclosed herein. Some embodiments provide synthetic or recombinant nucleic acid that encodes one or more of the polypeptides disclosed herein, and vectors (for example expression vectors) comprising the nucleic acid. Non-limiting examples of the method include synthetic ligation reassembly, random mutagenesis, targeted mutagenesis, optimized directed evolution system and/or saturation mutagenesis such as gene site saturation mutagenesis (GSSM), and any combination thereof. The term “variant” refers to polynucleotides or polypeptides in accordance with the description modified at one or more base pairs, codons, introns, exons, or amino acid residues (respectively) yet still retain the biological activity of a nuclease. Variants can be produced by methods such as by error-prone PCR, shuffling, site-directed mutagenesis, assembly PCR, sexual PCR mutagenesis, in vivo mutagenesis (phage-assisted continuous evolution, in vivo continuous evolution), cassette mutagenesis, recursive ensemble mutagenesis, exponential ensemble mutagenesis, site-specific mutagenesis, gene reassembly, gene site saturation mutagenesis, synthetic ligation reassembly, recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

Cloning vehicles comprising an expression cassette (such as a vector) can be used herein to express one or more of the nuclease polypeptides disclosed herein. The term “vector” as used herein encompasses any kind of cloning vehicles, such as but not limited to plasmids, phagemids, viral vectors (e.g., phages), bacteriophage, baculoviruses, cosmids, fosmids, artificial chromosomes, or and any other vectors specific for specific hosts of interest. Low copy number or high copy number vectors are also included. Foreign polynucleotide sequences usually comprise a coding sequence which may be referred to herein as “gene of interest”. The gene of interest may comprise introns and exons, depending on the kind of origin or destination of host cell. The cloning vehicle can be a viral vector, a plasmid, a phage, a phagemid, a cosmid, a fosmid, a bacteriophage, an artificial chromosome, or a combination thereof. The viral vector can comprise an adenovirus vector, a retroviral vector or an adeno-associated viral vector. The cloning vehicle can comprise a bacterial artificial chromosome (BAC), a plasmid, a bacteriophage Pl-derived vector (PAC), a yeast artificial chromosome (YAC), or a mammalian artificial chromosome (MAC). In some embodiments, the polynucleotide sequence encoding one or more nuclease polypeptides is integrated into a chromosome of the host cell in which the polynucleotide sequence is present, and thus the polynucleotide is a part of a chromosomal of the host cell. The host cell can be, for example, a bacterial cell, a mammalian cell, a fungal cell, a yeast cell, an insect cell, or a plant cell. In some embodiments, the polynucleotide sequence encoding one or more nuclease polypeptides is not located in the chromosome of the host cell.

Also provided herein are transformed host cells comprising nucleic acids or expression cassettes (such as vectors) or cloning vehicles comprising a nucleic acid sequence that encodes one or more of the nuclease polypeptides disclosed herein. Some embodiments provide a method for producing a recombinant polypeptide having nuclease activity, where the method comprises expressing a polynucleotide encoding one or more nuclease polypeptides disclosed herein under conditions that allow expression of at least one of the one or more nuclease polypeptides, thereby producing the recombinant polypeptide having nuclease activity. In some embodiments, the polynucleotide encoding one or more nuclease polypeptides disclosed herein is operably linked to a promoter. In some embodiments, the polypeptide is present in an expression vector. In some embodiments, the polynucleotide is present in the host cell to allow expression of the polypeptide. In some embodiments, the polynucleotide is present in a chromosome of the host cell to allow expression of the polypeptide. In some embodiments, the transformed host cell is a bacterial cell, a mammalian cell, a fungal cell, a yeast cell, an insect cell or a plant cell. In some embodiments, the transformed host cell is a cell from Escherichia coli.

Non-limiting examples of expression vectors include viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), Pi-based artificial chromosomes, yeast plasmids, yeast artificial chromosomes, and any other vectors specific for specific hosts of interest (such as bacillus, aspergillus and yeast). Nuclease-encoding DNA disclosed herein can be included in any one of a variety of expression vectors for expressing a nuclease polypeptide. Such vectors include chromosomal, nonchromosomal and synthetic DNA sequences. Many suitable vectors are known to those of skill in the art, and are commercially available, for example, pET-28a (Novagen). Depending on the desired use, low copy number or high copy number vectors can be used.

Codon optimization can be used to achieve high levels of protein expression in host cells. In some embodiments, codons in a nucleic acid encoding one or more of the nuclease polypeptides disclosed herein can be optimized to increase or decrease its expression in a host cell. For example, one or more of non-preferred or less preferred codons in the nucleic acid encoding the nuclease polypeptides can be replaced with one or more “preferred codons” encoding the same amino acid for a host cell of interest. As used herein, a “preferred codon” is a codon over-represented in coding sequences in genes in a host cell, and a “non-preferred or less preferred codon” is a codon under-represented in coding sequences in genes in the host cell. As an illustrated example, a codon optimized coding nucleic acid sequence of SEQ ID NO: 1 is disclosed herein as SEQ ID NO: 2.

Host cells for expressing the nucleic acids, expression cassettes and vectors in accordance with the present disclosure could be a eukaryotic cell or a prokaryotic cell, and could include bacteria, yeast, fungi, plant cells, insect cells and mammalian cells; and provides methods for optimizing codon usage in all of these cells, codon-altered nucleic acids and polypeptides made by the codon-altered nucleic acids. Exemplary host cells include gram negative bacteria and gram positive bacteria. Exemplary host cells also include eukaryotic organisms, such as various yeast and mammalian cells and insect cells. In some embodiments, the host cell is a cell from an organism selected from the group consisting of Pichia pastoris, Bacillus subtilis, Pseudomonas fluorescens, Myceliopthora thermophile fungus, Tricodermea reesei, Escherichia coli, Bacillus licheniformis, Aspergillus niger, Schizosaccharomyces pombe, and Sacaramyces cerevisiae. The nucleic acid encoding the nuclease polypeptide disclosed herein can be located on the genome of the host cell, for example be a part of a chromosome of the host cell. In some embodiments, the nucleic acid encoding the nuclease polypeptide is located on an expression vector separate from the genome of the host cell. Methods of producing a nuclease polypeptide disclosed herein can, in some embodiments, comprise, expressing a nucleic acid encoding the nuclease polypeptide under conditions that allow expression of the nuclease polypeptide, thereby producing the nuclease polypeptide. In some embodiments, the nucleic acid encoding the nuclease polypeptide is operably linked to an inducible promoter, for example a promoter inducible by changes in temperature and/or pH, and/or by the presence, absence or change in amount/concentration of a compound (e.g., IPTG, arabinose, tetracycline, steroids, and metal).

The description also includes nucleic acids and polypeptides optimized for expression in these organisms and species.

### Composition and Use

Methods are provided for degrading a polynucleotide using one or more of the nuclease polypeptides disclosed herein. In some embodiments, the method comprises contacting one or more polynucleotide molecules with one or more of the nuclease polypeptides disclosed herein, thereby degrading the polynucleotide molecules. The polynucleotide molecules can comprise DNA (e.g., single- or double-stranded DNA), RNA (e.g., single- or double-stranded DNA), or any combination thereof. The contacting can occur at various pH values, for example pH 4, pH 5, pH 6, pH 7, pH 8, pH 9, pH 10, pH 11, or a range between any two of these values. The contacting can also occur at various temperatures, for example, 10° C., 15° C., 20° C., 25° C., 30° C., 35° C., 40° C., 45° C., 50° C., 55° C., 60° C., 65° C., 70° C., or a range between any two of these values. The one or more polynucleotide molecules can be comprised in various objects, for example a composition for washing (e.g., textile, clothing, cotton, fabric, or any combination thereof), or an aqueous solution (e.g., a reaction mixture).

Provided herein are compositions and kits that comprise one or more nuclease polypeptides disclosed herein. The composition can be an enzyme composition, a detergent composition, a detergent additive, a food, a food supplement, a feed supplement, a feed, a pharmaceutical composition, a fermentation product, a fermentation intermediate, a fermentation downstream reaction mixture, a reaction mixture, or a combination thereof. In some embodiments, the reaction mixture is for protein expression or purification. The enzyme composition can, for example, comprise one or more of the nuclease polypeptide disclosed herein and a storage buffer. In some embodiments, the storage buffer comprises a pH buffering system (e.g., Tris-HCl) providing a pH of about 6.0-9.0, for example pH 6.0-7.0 or about pH 6.5. The storage buffer can, for example, include a stabilizing agent such as glycerol, at a concentration of at least about 5%, 10%, 20%, 30%, 40%, 50%, or more.

Also disclosed herein, the use of the polypeptides, nucleic acids, nucleic acid constructs or host cells disclosed herein in the manufacture of a pharmaceutical composition for preventing or treating a disease or disorder, such as wound, dental plaque; dental caries; periodontitis; native valve endocarditis; chronic bacterial prostatitis; otitis media; infections associated with medical devices such as artificial heart valves, artificial pacemakers, contact lenses, prosthetic joints, sutures, catheters, and arteriovenous shunts; infections associated with wounds, lacerations, sores and mucosal lesions such as ulcers; infections of the mouth, oropharynx, nasopharynx and laryngeal pharynx; infections of the outer ear; infections of the eye; infections of the stomach, small and large intestines; infections of the urethra and vagina; infections of the skin; intra-nasal infections, such as infections of the sinus; or a combination thereof. In some embodiments, the method for using the composition comprises contacting the composition with a wound, a laceration, a sore, a mucosal lesion, or any combination thereof. In some embodiments, the method for using the composition comprises contacting the composition with a medical device. In some embodiments, the method for using the composition comprises contacting the composition with skin, outer ear, eye, or a combination thereof. The compositions can be formulated in a variety of forms, such as tablets, gels, pills, implants, liquids, sprays, films, micelles, powders, food, feed pellets, a type of encapsulated form, or a combination thereof.

In some embodiments, the composition comprising one or more nuclease polypeptides disclosed herein is used to contact a surface comprising DNA substrate and to clean the surface.

In some embodiments, the nuclease polypeptides disclosed herein can be used alone, or in combination with one or more additional enzymes in an application or use. Some embodiments provide compositions comprising the nucleases and their variants disclosed herein. In some embodiments, the composition can further comprise one or more additional enzymes, or one or more additional components, or any combination thereof. The one or more additional enzymes can include, but are not limited to, one or more of nucleases, proteases, lipases, cutinases, amylases, carbohydrases, cellulases, pectinases, mannanases, arabinases, galactanases, xylanases, oxidases (e.g., laccases and peroxidases), and deoxyribonucleases (DNases).

Also provided are pharmaceutically acceptable prodrugs of the pharmaceutical compositions, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the agent). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in Bundgaard, Design of Prodrugs (Elsevier Press, 1985).

Also provided are pharmaceutically active metabolites of the pharmaceutical compositions, and uses of such metabolites in the methods of the description. A “pharmaceutically active metabolite” means a pharmacologically active product of metabolism in the body of a compound or salt thereof. Prodrugs and active metabolites of a compound may be determined using routine techniques known or available in the art. See, e.g., Bundgaard, Design of Prodrugs (Elsevier Press, 1985).

Any suitable formulation of the compounds described herein can be prepared. See, generally, Remington's Pharmaceutical Sciences. (2000) Hoover, J. E. editor, 20th edition. A formulation is selected to be suitable for an appropriate route of administration. Some routes of administration are oral, parenteral, by inhalation, topical, rectal, nasal, buccal, vaginal, via an implanted reservoir, or other drug administration methods. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, a-ketoglutarate, and a-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made.

### Method

Disclosed herein includes a method for degrading a polynucleotide, comprising contacting a polynucleotide molecule with one or more of the polypeptides disclosed herein, thereby degrading the polynucleotide molecule.

Also disclosed herein include a method for degrading DNA or RNA during cell lysis. The method comprises, in some embodiments, lysing a host cell of interest; and adding a polypeptide disclosed herein under conditions that allow degradation of DNA or RNA by the polypeptide.

Also disclosed herein include a method for degrading DNA or RNA during virus vector production. The method, in some embodiments, comprises culturing a host cell, wherein the host cell comprises a virus vector of interest; and expressing or adding one or more of the polypeptides disclosed herein under conditions that allow degradation of DNA or RNA other than the virus vector by the one or more polypeptides.

Also disclosed herein is a method for degrading polynucleotides during expression or production of a protein of interest. The method, in some embodiments, comprises culturing a host cell, wherein the host cell comprises a nucleic acid encoding a protein of interest; and expressing one or more of the nuclease polypeptides disclosed herein under conditions that allow degradation of polynucleotides by at least one of the one or more nuclease polypeptides. The polynucleotide molecules can comprise DNA, RNA, or any combination thereof. In some embodiments, at least one of the one or more nuclease polypeptides is not expressed by cells that express the protein of interest. In some embodiments, the one or more nuclease polypeptides are expressed by cells that do not express the protein of interest.

In these methods, the one or more nuclease polypeptides can be expressed from, for example, one or more expression vectors present in the host cell, or a nucleic acid sequence in a chromosome of the host cell. The expression of the protein of interest and/or the expression of the nuclease polypeptide can be inducible. For example, the coding sequence of the protein of interest, the coding sequence of the nuclease polypeptide, or both, can be operably linked with an inducible promoter. The inducible promoter can be, for example, induced by the presence, absence, and/or change in amount of one or more chemical or biological compounds, change in pH, temperature, osmolarity, ionic strength/concentration, or any combination thereof. Some embodiments provide a host cell comprising a nucleic acid encoding a protein of interest and a nucleic acid encoding one or more of the nuclease polypeptides disclosed herein.

### EXAMPLES

Aspects of the embodiments discussed above are further described in detail by reference to the following experimental examples. These examples are provided for illustrative purposes only and are not intended to be limiting unless otherwise specified. Thus, the present invention should by no means be construed as being limited to the following embodiments, but rather should be construed as including all the variations that become apparent as a result of the teaching provided herein. The methods and reagents used in the examples, unless otherwise stated, are those conventional in the art.

### Materials and Methods

### Protein Engineering

Based on crystal structure analyses of nuclease A from Serratia marcescens (PDB accession number: 1G8T), electrostatic surface reshaping was employed to improve salt tolerance of NucA by site specifically introducing positive charged residues to protein surface. 11 residues in wild type nuclease A corresponding to S74, N79, A95, T98, N101, A102, S137, D138, Q141, D149, D156 are mutated to positive residue lysine or arginine. Other fifteen variants (B0 to B14) were designed to investigate the combination effects of positive residues on multiple sites. Other variants with single site saturated mutations (B15 to B90) were designed to investigate contribution roles of different residues to high salt tolerance. The sequences of wild type NucA, B0, B1, B2, B3, B4 and B5 are listed as follow:

Wild NucA (SEQ ID NO: 1):
MRFNNKMLALAALLFAAQASADTLESIDNCAVGCPTGGSSNVSIVR
HAYTLNNNSTTKFANWVAYHITKDTPASGKTRNWKTDPALNPADTLAPA
DYTGANAALKVDRGHQAPLASLAGVSDWESLNYLSNITPQKSDLNQGAW
ARLEDQERKLIDRADISSVYTVTGPLYERDMGKLPGTQKAHTIPSAYWK
VIFINNSPAVNHYAAFLFDQNTPKGADFCQFRVTVDEIEKRTGLIIWAG
LPDDVQASLKSKPGVLPELMGCKN

B0: N79K, A95K, A102K, D149K;
B1: N79K, A95K, A102K, Q141K, D149K;
B2: N79K, A95K, N101K, A102K, Q141K, D149K;
B3: A95K, N101K, Q141K, D149K;
B4: S74K, N79K, A95K, T98K, N101K, Q141K, D149K;
B5: S74K, N79K, A95K, T98K, N101K, A102K, S137K, D138K, Q141K, D149K, D156R;
B6: N79R, A95R, A102R, D149R;
B7: A95K, A102K, D149K;
B8: N79K, A102K, D149K;
B9: N79K, A95K, D149K;
B10: N79K, A95K, A102K;
B11: A95R, A102R, D149R;
B12: N79R, A102R, D149R;
B13: N79R, A95R, D149R;
B14: N79R, A95R, A102R;
B15: N79K; B16: N79R; B17: N79A; B18: N79C; B19: N79D; B20: N79E; B21: N79F; B22: N79G; B23: N79H; B24: N79I; B25: N79L; B26: N79M; B27: N79P; B28: N79Q; B29: N79S; B30: N79T; B31: N79V; B32: N79W; B33: N79Y; B34: A95K; B35: A95R; B36: A95C; B37: A95D; B38: A95E; B39: A95F; B40: A95G; B41: A95H; B42: A95I; B43: A95L; B44: A95M; B45: A95N; B46: A95P; B47: A95Q; B48: A95S; B49: A95T; B50: A95V; B51: A95W; B52: A95Y; B53: A102K; B54: A102R; B55: A102C; B56: A102D; B57: A102E; B58: A102F; B59: A102G; B60: A102H; B61: A1021; B62: A102L; B63: A102M; B64: A102N; B65: A102P; B66: A102Q; B67: A102S; B68: A102T; B69: A102V; B70: A102W; B71: A102Y; B72: D149K; B73: D149R; B74: D149A; B75: D149C; B76: D149E; B77: D149F; B78: D149G; B79: D149H; B80: D149I; B81: D149L; B82: D149M; B83: D149N; B84: D149P; B85: D149Q; B86: D149S; B87: D149T; B88: D149V; B89: D149W; B90: D149Y

### Plasmids Preparation, Protein Expression and Purification

Genes encoding nuclease A mutants were codon optimized, synthesized (WuXi Biologics) and cloned into pET-28a (+) (Novagen), respectively. Plasmids were transformed into E. coli BL21 (DE3) respectively, the overexpressed proteins contain a C terminal 6-Histidine tag and can be purified by Ni-NTA resin (Cytiva). Positive clones were inoculated into 200 ml Luria-Bertani medium and cultured at 37° C. to an OD600 of 0.6-0.8, and protein expression was induced by adding isopropylthio-β-galactoside to final concentration of 0.4 mM, the cells was then transferred to 18° C. and cultured for another 20 hours. Cell culture was harvested by centrifuge at 3500 g, 4° C. for 20 min, cell pellet was resuspended using lysis buffer (20 Mm Tris, 300 mM NaCl, 10 mM imidazole, 5% v/v glycerol, pH8.0), and then cell lysis was performed using sonication, cell lysate was centrifuged at 16000 g, 4° C. for 20 min. The supernatant was then loaded on Ni-NTA column, and wash twice using wash buffer (20 Mm Tris, 300 mM NaCl, 40 mM imidazole, 5% v/v glycerol, pH8.0), the target protein was eluted using elution buffer (20 Mm Tris, 300 mM NaCl, 250 mM imidazole, 5% v/v glycerol, pH8.0). The elute was then further purified by gel filtration using HiLoad 16/600 Superdex 75 μg (Cytiva) and the protein was dialyzed into storage buffer (20 mM Tris-Cl, pH 8.0, 20 mM NaCl, 2 mM MgCl₂) and concentrated to about 1 mg/ml and supplemented with 50% glycerol for storage.

### Enzyme Specificity Assay

Enzyme specificity of wild type nuclease A and its mutant were performed by mixing 4U of enzymes with 1 μg of different substrates (including CHO cell total RNA, plasmid DNA-pET28 (a+), single stranded DNA and lambda DNA) in buffer containing different salt concentration, reactions were incubated at 37° C. for 30 min, and reactions were stopped by addition of 10 mM EDTA. The samples were loaded on 1% or 2% agrose gel and the digestion effects were analyzed by staining the gel using GelRed (Sigma-Aldrich)

### Enzyme Residual Activity Assay

Salmon sperm DNA was dissolved into reaction buffer (50 mM Tris, 2 mM MgCl2, pH 8.0) to a concentration of 1 mg/ml. Enzyme activity of wild type nuclease A and its mutants were performed by 2 ng different enzymes and 8 ul salmon sperm DNA (1 mg/ml) in different buffer with different salt concentration and pH, respectively. reactions were incubated at 37° C. for 30 min and reactions were stopped by adding 10 mM EDTA, and A260 absorption of each reaction was collected by NanoDrop (Thermo fisher). The relative enzyme activity was calculated by the following formula:

$$Relative enzyme activity = ( A 26 {0}_{sample} - A 26 {0}_{no enzyme} ) / ( A 26 {0}_{no salt} - A 26 {0}_{no enzyme} ) ,$$

A260ₛₐₘₚₗₑ stands for the A260 absorption of each sample; A260_{no enzyme} stands for the A260 absorption of control reaction without enzyme; A260_{no salt} stands for the A260 absorption of reaction in reaction buffer (50 mM Tris, 2 mM MgCl2, pH 8.0).

### Example 1

To investigate whether HighSalt NucA is a non-specific nuclease as the wild type nuclease A, different form nucleic acids, including super-coiled plasmid DNA, single-stranded DNA, linear double-stranded DNA (lambda phage genomic DNA), and total RNA, were digested by HighSalt NucA and wild type nuclease A in buffer containing different NaCl concentration. With or without further explanation, mutant B0 is equivalent to the term “HighSalt NucA” in this and the following Example.

### HighSalt NucA is Able to Digest Linear Double-Stranded DNA at High Salt Concentration

Lambda phage genomic DNA (New England Biolabs, Cat. No. N3011S) was incubated with HighSalt NucA and wild type nuclease A respectively. The results indicated that the digestion by wild type nuclease A was significantly inhibited in buffer containing >200 mM NaCl (FIG. 2). However, HighSalt NucA is able to effectively digest lambda DNA in buffer containing 500 mM NaCl (FIG. 2).

### HighSalt NucA is Able to Digest Supercoiled Plasmid DNA at High Salt Concentration

Plasmid pET28a (+) (Novagen) was incubated with HighSalt NucA and wild type nuclease A respectively. The results are similar to the lambda DNA digestion assays. The digestion by wild type nuclease A was significantly inhibited in buffer containing >200 mM NaCl, whereas HighSalt NucA is able to effectively digest plasmid DNA at salt concentration upto 500 mM (FIG. 3).

### HighSalt NucA is Able to Digest RNA at High Salt Concentration

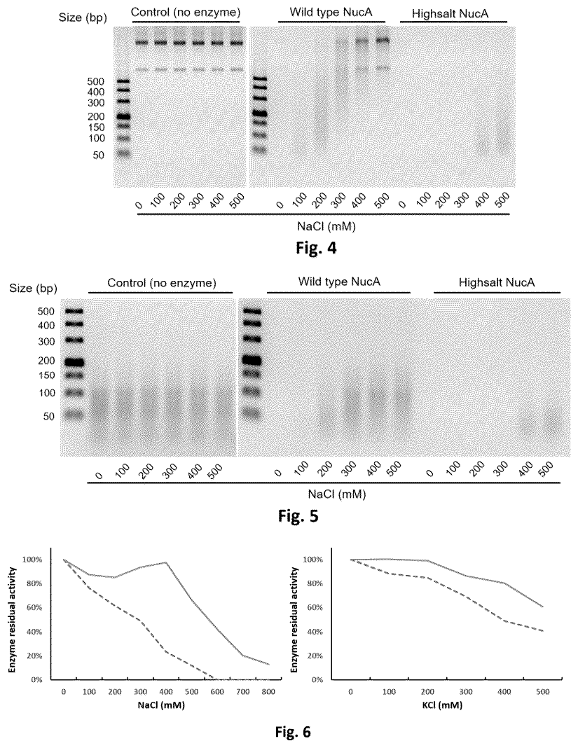

Total RNA from CHO cells (WuXi Biologics Co., Ltd), extracted using RNeasy Plus Mini Kit (QIAGEN, Cat. No. 74134), was incubated with HighSalt NucA and wild type nuclease A respectively. The most abundant RNA in total RNA is ribosomal RNA, which containing double-stranded and single-stranded portion in different region. The digestion results also indicated that wild type nuclease A was greatly inhibited when salt concentration is over 200 mM (FIG. 4). HighSalt NucA is able to effectively digest RNA at salt concentration upto 500 mM (FIG. 4).

### HighSalt NucA is Able to Digest Single-Stranded DNA at High Salt Concentration

Single-stranded DNA (120-130 nt) was synthesized and incubated with HighSalt NucA and wild type nuclease A respectively. The results indicated that the digestion by wild type nuclease A was significantly inhibited in buffer containing >200 mM NaCl (FIG. 5). However, HighSalt NucA is also able to effectively digest single-stranded DNA in buffer containing 500 mM NaCl (FIG. 5).

### Conclusion

Wild type Serratia marcescens nuclease A is a non-specific nuclease that digest nucleic acids in different forms. By performing enzyme specificity assays, we found that engineered HighSalt NucA maintains its non-specific catalytic activity towards different forms of nucleic acids. In addition, HighSalt NucA displays significantly higher salt tolerance than wild type nuclease A, and is still effective under high salt concentration of 500 mM NaCl.

### Example 2

To investigate inhibition effects of different salts towards wild type nuclease A and HighSalt NucA, enzyme residual activity assays were performed by incubating salmon sperm DNA with wild type nuclease A and HighSalt NucA in buffer containing different concentration of monovalent-(NaCl, KCl), divalent-(MgCl₂, MnCl₂, (NH₄) ₂SO₄), trivalent-(Na₂HPO₄) salt, respectively.

### Inhibition Effects of Monovalent Salts on HighSalt NucA

Wild type nuclease A is sensitive to the concentration of both NaCl and KCl, >300 mM monovalent sat concentration dramatically decrease the activity of wild type nuclease below 60% A (FIG. 6). However, engineered HighSalt NucA maintain >60% enzyme activity at salt concentration of 500 mM (FIG. 6). Thus, the engineered HighSalt NucA possesses higher tolerance against monovalent salts.

### Inhibition Effects of Divalent Salts on HighSalt NucA

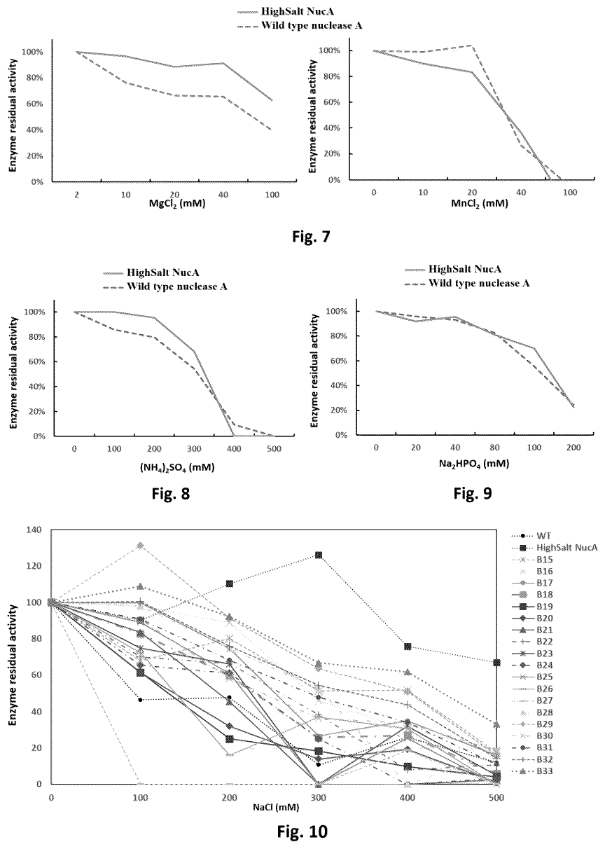

Divalent salts provide stronger ionic strength in solution, we also investigate the divalent ions effects on nuclease A and HighSalt NucA. Similar to the performance of HighSalt NucA in monovalent salt buffer, the results imply that HighSalt NucA maintain 60% enzyme activity at MgCl₂ concentration of 400 mM. HighSalt NucA tolerate MgCl₂ better than wild type nuclease A (FIG. 7). Furthermore, HighSalt NucA also tolerate (NH₄) ₂SO₄ slightly better than wild type nuclease A (FIG. 8). However, inhibition effects of MnCl₂ seems to be greater on HighSalt NucA than on wild type nuclease A (FIG. 7).

### Inhibition Effects of Trivalent Salts on HighSalt NucA

Due to chelating effect, metal dependent nuclease is sensitive to PO₄³⁻. Both HighSalt NucA and nuclease A perform similar in PO₄³⁻ buffer, the enzyme activity of both enzymes are significantly inhibited by >100 mM PO₄³⁻ (FIG. 9).

### Conclusion

The enzyme residual activity assays imply that HighSalt NucA tolerate higher concentration of NaCl, KCl and (NH₄) ₂SO₄ than wild type nuclease A. The engineering of nuclease A by introduce positive residues to specific surface indeed improve its high salt tolerance.

### Example 3

To investigate salt tolerant contribution of single residue mutations on N79, A95, A102 and D149, each residue was mutated to other 19 amino acids respectively. Activity of each mutants under different salt concentration were analyzed.

Mutations on N79 and their Contribution to Salt Tolerance

Residue N79 was mutated to other 19 amino acids respectively. Mutants were purified and the salt tolerance of mutants were analyzed. In addition to mutations of positive residues K and R, many other mutations, such as A, C, G, Q, S, T, V, W, Y perform better salt tolerance than wild type enzyme (FIG. 10 and Table 1).

TABLE 1
The salt tolerance of wild type NucA, HighSalt NucA
and nineteen mutants with single mutation on N79 residue.

| Residual | NaCl (mM) | | | | | |
|---|---|---|---|---|---|---|
| Activity (%) | 0 | 100 | 200 | 300 | 400 | 500 |
| WT | 100 | 46.6 | 47.9 | 10.8 | 25.8 | 12.0 |
| HighSalt | 100 | 90.2 | 110.2 | 126.2 | 75.8 | 66.9 |
| NucA (B0) | | | | | | |
| B15 | 100 | 67.9 | 80.1 | 51.3 | 51.8 | 17.7 |
| B16 | 100 | 98.1 | 88.9 | 46.2 | 30.4 | 19.3 |
| B17 | 100 | 99.7 | 74.6 | 26.6 | 35.0 | 15.8 |
| B18 | 100 | 82.8 | 59.7 | 25.7 | 27.0 | 5.9 |
| B19 | 100 | 61.4 | 25.1 | 18.3 | 9.8 | 4.1 |
| B20 | 100 | 61.3 | 32.0 | 13.9 | 19.2 | 0.9 |
| B21 | 100 | 83.4 | 45.5 | 0 | 0 | 2.4 |
| B22 | 100 | 70.0 | 66.3 | 38.4 | 7.9 | 10.6 |
| B23 | 100 | 74.7 | 66.0 | 0 | 32.0 | 5.0 |
| B24 | 100 | 65.3 | 61.1 | 25.1 | 0 | 3.3 |
| B25 | 100 | 89.0 | 60.8 | 0 | 25.4 | 0 |
| B26 | 100 | 72.2 | 16.0 | 37.1 | 30.6 | 0 |
| B27 | 100 | 0 | 0 | 0 | 18.7 | 0 |
| B28 | 100 | 98.0 | 74.6 | 52.7 | 0 | 17.0 |
| B29 | 100 | 131.4 | 91.2 | 63.6 | 51.1 | 15.9 |
| B30 | 100 | 90.4 | 58.3 | 36.2 | 33.8 | 18.4 |
| B31 | 100 | 90.8 | 68.2 | 47.9 | 34.1 | 11.0 |
| B32 | 100 | 100.4 | 75.9 | 54.3 | 43.8 | 14.2 |
| B33 | 100 | 108.9 | 92.2 | 66.7 | 61.8 | 33.2 |

Mutations on A95 and their Contribution to Salt Tolerance

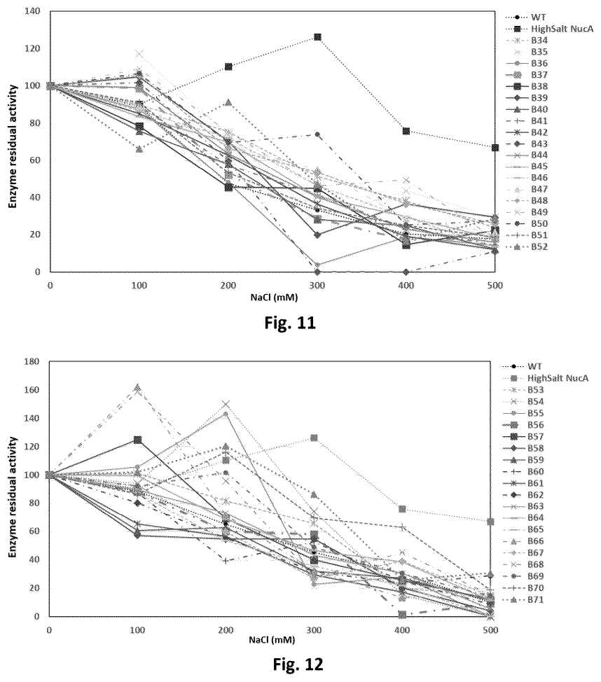

Residue A95 was mutated to other 19 amino acids respectively. Mutants were purified and the salt tolerance of mutants were analyzed. In addition to mutations of positive residues K and R, other mutations, such as F, N, P, Q, S, T, V, Y display positive effect on improving salt tolerance of mutants (FIG. 11 and Table 2).

TABLE 2
The salt tolerance of wild type NucA, HighSalt NucA
and nineteen mutants with single mutation on A95 residue.

| Residual | NaCl (mM) | | | | | |
|---|---|---|---|---|---|---|
| Activity (%) | 0 | 100 | 200 | 300 | 400 | 500 |
| WT | 100 | 88.9 | 47.6 | 33.1 | 20.3 | 17.6 |
| HighSalt | 100 | 90.2 | 110.2 | 126.2 | 75.8 | 66.9 |
| NucA (B0) | | | | | | |
| B34 | 100 | 89.9 | 75.3 | 51.2 | 38.0 | 23.8 |
| B35 | 100 | 117.1 | 73.8 | 28.6 | 43.6 | 29.6 |
| B36 | 100 | 87.5 | 47.9 | 3.7 | 18.8 | 14.1 |
| B37 | 100 | 98.7 | 52.1 | 28.5 | 17.2 | 17.9 |
| B38 | 100 | 78.3 | 45.4 | 44.9 | 14.6 | 22.2 |
| B39 | 100 | 104.7 | 70.3 | 19.9 | 36.3 | 29.2 |
| B40 | 100 | 75.6 | 57.9 | 28.1 | 24.7 | 12.1 |
| B41 | 100 | 89.2 | 57.4 | 41.3 | 23.3 | 14.3 |
| B42 | 100 | 85.0 | 62.5 | 36.4 | 18.9 | 11.9 |
| B43 | 100 | 101.7 | 59.6 | 0 | 0 | 10.9 |
| B44 | 100 | 98.9 | 63.5 | 40.3 | 22.8 | 15.7 |
| B45 | 100 | 83.4 | 69.8 | 40.6 | 29.4 | 17.8 |
| B46 | 100 | 86.5 | 71.9 | 46.5 | 29.2 | 13.3 |
| B47 | 100 | 89.1 | 67.9 | 55.2 | 28.7 | 21.1 |
| B48 | 100 | 105.7 | 66.3 | 53.4 | 36.5 | 25.8 |
| B49 | 100 | 109.0 | 75.3 | 46.1 | 49.3 | 17.9 |
| B50 | 100 | 106.6 | 69.5 | 73.8 | 25.2 | 27.4 |
| B51 | 100 | 90.9 | 53.3 | 34.1 | 24.8 | 18.5 |
| B52 | 100 | 66.1 | 91.3 | 47.0 | 16.7 | 28.6 |

Mutations on A102 and their Contribution to Salt Tolerance

Residue A102 was mutated to other 19 amino acids respectively. Mutants were purified and the salt tolerance of mutants were analyzed. Among these mutations, mutants containing K, R, T, V, W and Y possess better salt tolerance than wild type enzyme (FIG. 12 and Table 3).

TABLE 3
The salt tolerance of wild type NucA, HighSalt NucA and
nineteen mutants with single mutation on A102 residue.

| Residual | NaCl (mM) | | | | | |
|---|---|---|---|---|---|---|
| Activity (%) | 0 | 100 | 200 | 300 | 400 | 500 |
| WT | 100 | 88.4 | 65.0 | 45.2 | 30.3 | 9.9 |
| HighSalt | 100 | 90.2 | 110.2 | 126.2 | 75.8 | 66.9 |
| NucA (B0) | | | | | | |
| B53 | 100 | 101.0 | 81.6 | 65.6 | 20.3 | 13.9 |
| B54 | 100 | 94.0 | 149.8 | 74.0 | 15.1 | 0 |
| B55 | 100 | 105.6 | 143.1 | 22.8 | 27.7 | 0 |
| B56 | 100 | 87.9 | 60.0 | 57.9 | 1.4 | 9.4 |
| B57 | 100 | 124.8 | 69.0 | 39.8 | 25.5 | 12.2 |
| B58 | 100 | 57.0 | 54.5 | 54.5 | 20.1 | 4.0 |
| B59 | 100 | 60.3 | 62.4 | 31.9 | 27.0 | 11.3 |
| B60 | 100 | 87.4 | 39.2 | 52.6 | 24.8 | 8.8 |
| B61 | 100 | 65.3 | 56.5 | 29.4 | 17.2 | 0 |
| B62 | 100 | 80.1 | 55.1 | 31.7 | 23.3 | 28.7 |
| B63 | 100 | 89.3 | 72.7 | 43.0 | 30.4 | 5.6 |
| B64 | 100 | 99.4 | 68.3 | 46.3 | 38.5 | 11.2 |
| B65 | 100 | 87.1 | 53.3 | 35.3 | 21.7 | 0 |
| B66 | 100 | 162.2 | 71.4 | 32.1 | 13.3 | 14.9 |
| B67 | 100 | 92.1 | 59.6 | 26.8 | 38.9 | 14.5 |
| B68 | 100 | 158.7 | 95.8 | 27.8 | 45.3 | 15.1 |
| B69 | 100 | 92.0 | 101.6 | 48.8 | 30.6 | 14.3 |
| B70 | 100 | 86.3 | 116.0 | 69.4 | 62.9 | 18.8 |
| B71 | 100 | 101.9 | 120.2 | 86.3 | 25.8 | 30.7 |

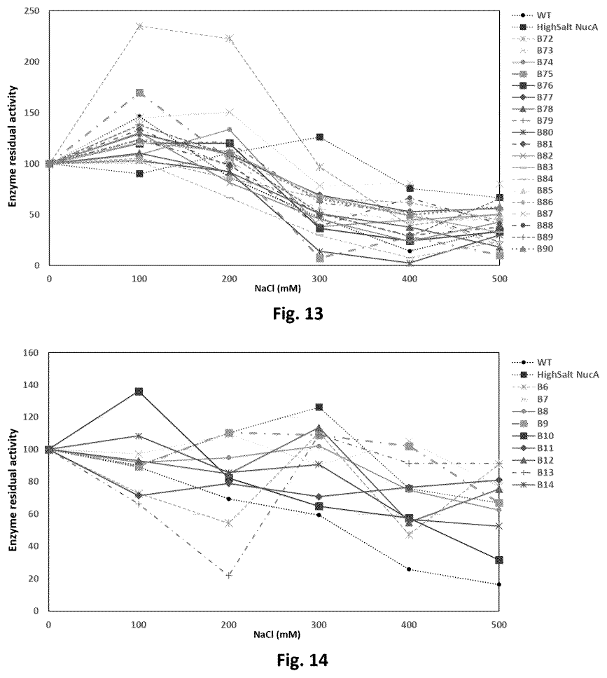

Mutations on D149 and their contribution to salt tolerance Residue D149 was mutated to other 19 amino acids respectively. Mutants were purified and the salt tolerance of mutants were analyzed. Reducing acidic surface at this site by mutating to most of other residues improve salt tolerance of enzyme, the promising resides for this site can be replaced by A, F, H, K, N, Q, R, S, T, V, W, Y (FIG. 13 and Table 4).

TABLE 4
The salt tolerance of wild type NucA,
HighSalt NucA and nineteen
mutants with single mutation on D149 residue.

| Residual | NaCl (mM) | | | | | |
|---|---|---|---|---|---|---|
| Activity (%) | 0 | 100 | 200 | 300 | 400 | 500 |
| WT | 100 | 146.3 | 86.8 | 47.1 | 14.2 | 35.1 |
| HighSalt | 100 | 90.2 | 110.2 | 126.2 | 75.8 | 66.9 |
| NucA (B0) | | | | | | |
| B72 | 100 | 235.1 | 222.9 | 97.0 | 39.2 | 50.8 |
| B73 | 100 | 144.9 | 150.5 | 78.4 | 81.0 | 80.5 |
| B74 | 100 | 109.0 | 133.5 | 38.0 | 44.7 | 49.9 |
| B75 | 100 | 169.7 | 104.0 | 7.3 | 28.2 | 10.1 |
| B76 | 100 | 119.9 | 120.0 | 36.5 | 24.0 | 33.3 |
| B77 | 100 | 129.4 | 108.8 | 69.2 | 52.7 | 56.1 |
| B78 | 100 | 110.3 | 91.7 | 50.6 | 37.5 | 18.0 |
| B79 | 100 | 119.9 | 121.0 | 61.6 | 52.6 | 23.1 |
| B80 | 100 | 102.9 | 92.2 | 13.8 | 2.2 | 29.8 |
| B81 | 100 | 133.4 | 97.6 | 50.7 | 29.5 | 37.5 |
| B82 | 100 | 130.2 | 81.4 | 45.4 | 23.6 | 42.6 |
| B83 | 100 | 120.8 | 111.9 | 68.0 | 48.9 | 32.5 |
| B84 | 100 | 102.5 | 66.6 | 29.6 | 7.6 | 23.3 |
| B85 | 100 | 121.3 | 102.1 | 55.8 | 43.5 | 45.7 |
| B86 | 100 | 105.2 | 85.5 | 66.3 | 61.7 | 44.8 |
| B87 | 100 | 137.9 | 107.0 | 66.0 | 51.6 | 19.6 |
| B88 | 100 | 123.2 | 99.2 | 38.5 | 66.5 | 41.0 |
| B89 | 100 | 138.1 | 108.3 | 51.7 | 28.8 | 65.3 |
| B90 | 100 | 123.6 | 111.6 | 65.0 | 49.5 | 57.8 |

### Combination Effect of Positive Residues on Salt Tolerance

Based on above results, single residue mutations, especial K and R mutation, improve salt tolerance of enzyme, however the effect is limited. Thus, we hypothesize that the salt tolerance can be enhanced by combination of mutations. We chose N79, A95, A102, D149, and mutated these residues by combination, and the results indicate a significant improvement of salt tolerance than that of wild type enzyme and enzymes with single mutation (FIG. 14 and Table 5). In addition, the results also prove another hypothesis that enzyme activity improvement under high salt buffer is depending on the balance of binding and substrate releasing.

TABLE 5
The salt tolerance of wild type NucA, HighSalt NucA and
mutants with mutations of combination positive residues.

| Residual | NaCl (mM) | | | | | |
|---|---|---|---|---|---|---|
| Activity (%) | 0 | 100 | 200 | 300 | 400 | 500 |
| WT | 100 | 88.9 | 69.4 | 59.3 | 25.8 | 16.3 |
| HighSalt | 100 | 90.2 | 110.2 | 126.2 | 75.8 | 66.9 |
| NucA (B0) | | | | | | |
| B6 | 100 | 72.8 | 54.3 | 110.5 | 47.2 | 91.0 |
| B7 | 100 | 97.4 | 109.5 | 90.8 | 105.3 | 77.0 |
| B8 | 100 | 92.0 | 94.8 | 102.0 | 74.6 | 62.4 |
| B9 | 100 | 89.5 | 110.5 | 108.8 | 102.0 | 66.8 |
| B10 | 100 | 136.1 | 82.3 | 64.8 | 57.4 | 31.5 |
| B11 | 100 | 71.3 | 78.9 | 70.7 | 76.4 | 81.0 |
| B12 | 100 | 92.8 | 84.8 | 113.6 | 54.7 | 75.3 |
| B13 | 100 | 66.2 | 21.9 | 109.1 | 91.3 | 91.3 |
| B14 | 100 | 108.3 | 85.5 | 90.6 | 56.7 | 52.5 |

### Conclusion

Besides to mutation of positive residues K and R, other residues also contribute improve effect to salt tolerance. However, single residue mutation is insufficient for the best performance, thus multiple sites mutation, especial multiple site K/R mutations significantly improve enzyme activity under high salt condition.

1. A polypeptide derived from Serratia marcescens nuclease A, and having high salt tolerance, the polypeptide comprises one or more mutations so that the polypeptide possesses more positive charged surface area in its three-dimensional structure.

2. The polypeptide according to claim 1,
wherein, the polypeptide comprises:
(a) an amino acid sequence having at least 70% identity to SEQ ID NO: 1 or the mature polypeptide thereof and has mutations at 1, 2, 3, 4, 5, 6, 7 or more or all sites of S74, N79, A95, T98, N101, A102, S137, D138, Q141, D149, D156, or
(b) an amino acid sequence having at least 70% sequence identity to the sequence of (a) and has the mutation at the site as described in (a) and retains nuclease activity.

3. The polypeptide according to claim 2, the polypeptide comprises an amino acid sequence having at least 70% identity to SEQ ID NO: 1 or the mature polypeptide thereof and has a mutation at (1) A95 and at least 3 sites selected from the group consisting of S74, N79, T98, N101, A102, S137, D138, Q141, D149 D156; or (2) D149 and at least 3 sites selected from the group consisting of S74, N79, A95, T98, N101, A102, S137, D138, Q141, D156.

4. The polypeptide according to claim 2, the polypeptide comprises an amino acid sequence having at least 70% identity to SEQ ID NO: 1 or the mature polypeptide thereof and has mutations at at least three sites selected from the group consisting of N79, A95, A102, and D149 and optionally at least one site selected from the group consisting of S74, T98, N101, S137, D138, Q141, D156.

5. The polypeptide according to claim 2, the polypeptide comprises an amino acid sequence having at least 70%, sequence identity to SEQ ID NO: 1 or the mature polypeptide thereof, where the polypeptide has nuclease activity under high solution ionic strength, and where the polypeptide comprises one or more mutations of (1) N79K or N79R, (2) A95K or A95R, (3) A102K or A102R, (4) D149K or D149R, (5) S74K, (6) T98K, (7) N101K, (8) S137K, (9) D138K, (10) Q141K, and (11) D156R.

6. A composition or kit comprising one or more of the polypeptides according to claim 1,
preferably, the composition is a reaction mixture, a detergent composition, a detergent additive, a food, a food supplement, a feed supplement, a feed, a pharmaceutical composition, a fermentation product, a fermentation intermediate, a fermentation downstream reaction mixture, or a combination thereof.

7. A nucleic acid comprising:
(1) the sequence that encodes one or more of the polypeptides according to claim 1, or a complementary sequence thereof, or
(2) a sequence with at least 50%, 60%, 70%, 80%, or 90% identity with (1).

8. A nucleic acid construct comprising the polynucleotide sequence of the nucleic acid according to claim 7.

9. A reaction mixture, wherein the reaction mixture comprises: (a) one or more of the polypeptides according to claim 1, (b) one or more nucleic acid molecules, and (c) an aqueous solution wherein the polypeptide hydrolyzes the one or more nucleic acid molecules.

10. A cell comprising one or more of the polypeptides according to claim 1, one or more nucleic acid that encodes the polypeptides, one or more nucleic acid constructs comprising the polynucleotide sequence of the nucleic acid, or a combination thereof.

11. A method of producing a polypeptide having nuclease activity under high solution ionic strength, comprising: expressing the nucleic acid that encodes any one of the polypeptides according to claim 1 under conditions that allow expression of the polypeptide, thereby producing recombinant polypeptide having nuclease activity, wherein the nucleic acid is operably linked to a promoter.

12. A method for degrading a polynucleotide, comprising contacting a polynucleotide molecule with one or more of the polypeptides according to claim 1, thereby degrading the polynucleotide molecule.

13. A method for degrading DNA or RNA during cell lysis, comprising: lysing a host cell of interest; and contacting the lysate with one or more of the polypeptides according to claim 1 herein under conditions that allow degradation of DNA or RNA by the polypeptide.

14. A method for degrading DNA or RNA during virus vector production, comprising: culturing a host cell, wherein the host cell comprises a virus vector of interest; and expressing or adding one or more of the polypeptides according to claim 1 under conditions that allow degradation of DNA or RNA other than the virus vector by the one or more polypeptides.

15. A method for degrading DNA or RNA during protein production, comprising: culturing a host cell, wherein the host cell comprises a nucleic acid encoding a protein of interest; and expressing or adding one or more of the polypeptides according to claim 1 under conditions that allow degradation of DNA or RNA by the one or more polypeptides.

16. A method for degrading DNA or RNA in a protein production mixture, comprising: culturing a host cell which comprises a nucleic acid encoding a protein of interest; and expressing or adding one or more of the polypeptides according to claim 1 under conditions that allow degradation of DNA or RNA by the polypeptide.

17. The polypeptide according to claim 1, wherein, the polypeptide has at least 60% nuclease activity under a solution ionic strength more than 200 mM as compared to the nuclease having the sequence of SEQ ID NO: 1.

18. The polypeptide according to claim 2, wherein, the mutation of N79, A95, A102, D149 is each independently a mutation to a non polar amino acids such as alanine, valine, isoleucine, proline, phenylalanine, tryptophan methionine, or glycine, or to an uncharged polar amino acid such as serine, threonine, glutamine, tyrosine, or cysteine, or to a polar amino acid with positive charges such as histidine, lysine or arginine.

19. The polypeptide according to claim 2, wherein, the mutation of S74, N79, A95, T98, N101, A102, S137, D138, Q141, D149, D156 is a mutation to a positive amino acid residue, preferably histidine, lysine or arginine.

20. The composition or kit according to claim 6, wherein, the composition is a reaction mixture, a detergent composition, a detergent additive, a food, a food supplement, a feed supplement, a feed, a pharmaceutical composition, a fermentation product, a fermentation intermediate, a fermentation downstream reaction mixture, or a combination thereof.


SEQUENCE LISTING (2 sequences)

SEQ ID NO: 1 - protein, 266 aa, Serratia marcescens
MRFNNKMLALAALLFAAQASADTLESIDNCAVGCPTGGSSNVSIVRHAYTLNNNSTTKFANWVAYHITKDTPASGKTRNWKTDPALNPADTLAPADYTGANAALKVDRGHQAPLASLAGVSDWESLNYLSNITPQKSDLNQGAWARLEDQERKLIDRADISSVYTVTGPLYERDMGKLPGTQKAHTIPSAYWKVIFINNSPAVNHYAAFLFDQNTPKGADFCQFRVTVDEIEKRTGLIIWAGLPDDVQASLKSKPGVLPELMGCKN

SEQ ID NO: 2 - DNA, 804 bp, synthetic construct
  source 1..804: mol_type = other DNA
atgcggtttaataacaagatgttggcactggcggcgctgttattcgcggctcaggcatccgctgatacccttgaatcgattgataactgcgcggtaggctgtccaactggtggtagtagtaacgttagcatagttagacacgcatacaccctgaataataatagcacgactaaattcgctaactgggtagcctatcatataactaaagacacgccggctagtggtaaaacccgtaactggaaaactgacccggcgttaaacccggctgatactctggcaccggcggattacaccggcgctaacgccgccctaaaggtagaccgtggccaccaggcgccgctggcgagcctggcgggtgtgagcgattgggaatccctgaactacctgagcaacatcaccccgcagaaatccgatctgaaccagggtgcgtgggcgcgtctggaagatcaggaacgtaaactgatcgatcgtgcggatatctctagcgtgtacaccgttaccggcccgctgtacgaacgtgatatgggtaaactgccgggtacccagaaagcgcacaccatcccgagcgcgtactggaaagttatcttcatcaacaacagcccggcggttaaccactacgcggcgttcctgttcgatcagaacaccccgaaaggtgcggatttctgccagttccgtgttaccgttgatgaaatcgagaaacgtaccggtctgatcatctgggcgggtctgccggatgatgttcaggcgagcctgaaatctaaaccgggtgttctgccggaactgatgggctgcaaaaactaatga